(19) United States
(12) Patent Application Publication
(10) Pub. No.: US 2024/0197228 A1
(43) Pub. Date: Jun. 20, 2024

(54) ELECTRODE ARRANGEMENT FOR MEASURING BIOPOTENTIALS ON A PERSON'S HEAD

(71) Applicant: Fraunhofer-Gesellschaft zur Förderung der angewandten Forschung e.V., München (DE)

(72) Inventors: Karen Insa WOLF, Ilmenau (DE); Wiebke PÄTZOLD, Ilmenau (DE); Carlos Filipe DA SILVA SOUTO, Ilmenau (DE); Stefan DEBENER, Ilmenau (DE); Paul MARINA, Stadtlohn (DE)

(21) Appl. No.: 18/594,584

(22) Filed: Mar. 4, 2024

Related U.S. Application Data
(63) Continuation of application No. PCT/EP2022/074614, filed on Sep. 5, 2022.

(30) Foreign Application Priority Data
Sep. 6, 2021 (DE) 10 2021 209 778.2

(51) Int. Cl. A61B 5/256; A61B 5/00; A61B 5/16; A61B 5/259; A61B 5/291

(52) U.S. Cl. CPC A61B 5/256; A61B 5/16; A61B 5/259; A61B 5/291; A61B 5/4088; A61B 5/4094; A61B 5/4806

(57) ABSTRACT

Device with an electrode arrangement with a plurality of electrodes for measuring electric signals on a face surface of a head of a user and behind an ear of the user; and with a carrier structure. The carrier structure at least partially defines a relative position of the electrodes to one another, and the carrier structure is shape-stable within a tolerance range without external force exerted by a user and deformable under external force exerted by the user, in order to change a distance between two adjacently arranged electrodes and/or to area-comprehensively adapt to face and head structures. The carrier structure is configured to position at least a part of the electrode arrangement on the face surface and behind the ear.

### CROSS-REFERENCE TO RELATED APPLICATIONS

This application is a continuation of copending International Application No. PCT/EP2022/074614, filed Sep. 5, 2022, which is incorporated herein by reference in its entirety, and additionally claims priority from German Application No. 10 2021 209 778.2, filed Sep. 6, 2021, which is also incorporated herein by reference in its entirety.

### TECHNICAL FIELD

Embodiments according to the invention relate to a device having an electrode arrangement, a system having the device and having an evaluation arrangement, and a method for producing the device, as well as a method for using the device. The electrode arrangement is configured, e.g., for measuring electric signals on a head of a human.

Embodiments according to the invention relate, inter alia, to self-applicable electrode grid configurations, i.e. carrier structures having an electrode arrangement, for non-invasive long term measuring of biopotentials on a human.

### BACKGROUND OF THE INVENTION

For gathering electrophysiological signals, in particular by means of an electroencephalogram (EEG), electrooculogram (EOG), electrocardiogram (ECG or EKG), and electromyogram (EMG), electrodes are brought into contact with the skin of a human using a gel (wet electrodes) or also without a gel (dry electrodes) in order to measure an electric activity generated by the brain and muscle cells. The application of the electrodes, in particular the EEG electrodes on the head, in combination with a sufficiently high signal quality has been complicated, particularly in the case of wet electrodes, which are therefore not suitable for self-application by a layperson. Dry electrodes are rather unsuitable for EEG measurements as they have a high transition impedance, and the pressure on the scalp may cause paresthesia and headaches. In the field of EKG, so-called solid gel electrodes are available as wet electrodes, which can be easily affixed to the skin like an adhesive bandage as a single electrode. In addition to the electrolyte effect, the moderately viscous gel lens also has adhesive properties, so that the electrode adheres to the skin better than is the case with wet electrodes having gel applied or dry electrodes. For detecting the EEG and EOG, positions on the head/in the face are to be used (close to the eyes or rather close to the brain as the signal source), wherein the positions should be met within a tolerance of a few cm in order to be able to record the specific signal of brain activity and eye movement.

Polysomnography (PSG) is the diagnostic standard method for detecting sleep phases. Here, various biosignals are continuously recorded during sleep, usually in-hospital overnight in a sleep laboratory. This includes the collection of EEG, EOG, EMG, EKG, respiratory flow, breathing effort, oxygen saturation, body position, and video data. Usually, positions as represented in FIG. 14 are used as electrode positions for collecting EEG, EOG, and EMG. Small deviations in configuration are possible. FIG. 14 thus shows a conventional electrode placement in a polysomnography.

EOG_R and EOG_L detect the eye movements. GND and REF are used for measuring the EEG with a difference amplifier in which potential differences regarding REF and GND are identified. F4, F3, C4, C3, O1, O2 are positions of the EEG electrodes, following the 10-20 system common in the context of EEG, in which one occipital, one central, and one frontal position each are needed determining the sleep phases, and are supplemented during measurement by one alternative position each. M1 and M2 are used for classic re-referencing to the mastoid. The electrodes on the chin (EMG1, EMG_REF, EMG2) are used for detecting muscle movements or muscle tension. The signals of electrodes F4, F3, C4, C3, O1, O2 are used in combination and relating to REF and GND as so-called EEG channels for determining the sleep phases. The placement of all electrodes is very time consuming and only possible for trained personnel. For EEG monitoring outside of the hospital and research laboratory, in which case people apply electrodes themselves, to date, only dry electrode systems are available for fixed electrode configurations. In the field of sleep monitoring, there are dry electrode solutions, which, however, do not map an EOG or EMG in the form proposed here (cf. e.g. https://dreem.com/, 22.7.2021 [Arnal, P. J., Thorey, V., Ballard, M. E., Hernandez, A. B., Guillot, A., Jourde, H., & Sauvet, F. (2019); “The Dreem headband as an alternative to polysomnography for EEG signal acquisition and sleep staging.”; BioRxiv, 662734] or https://www.philips.de/c-e/hs/sleep-solutions/smartsleep-advocacy, 22.7.2021). Wet electrodes for a high signal quality are currently affixed exclusively by trained personnel and not by untrained people alone. Additionally, there are helmet/headphone solutions using sponges that are to be kept moist.

There are printed electrode systems on the market:

(1) cEEGrid
With these electrodes, the gel is applied directly prior to the measurement, it is not possible to apply the grid yourself, as a trained person is needed for this. The carrier material of the printed electrode is flexible only in one direction, so that it does not adapt well to a three dimensional shape (e.g. bone projection on the head). It has sharp edges, which may impair the wear comfort in the ear area.
(cf. https://tmsi.wiljekoffie.dev/product/ceegrid (21.7.2021);
Debener, S., Emkes, R., De Vos, M. & Bleichner, M.; “Unobtrusive ambulatory EEG using a smartphone and flexible printed electrodes around the ear.”; Sci. Rep. 5, 16743 (2015); and
Bleichner, M. G. & Debener, S.; “Concealed, unobtrusive ear-centered EEG acquisition: cEEGrids for transparent EEG.”; Front Hum Neurosci. 11, (2017))
   (2) xTrodes
These are dry electrodes and it is unknown whether or to what extent the grid may be applied by oneself.
(cf. https://xtrodes.com/; WO 2017/090050 A1; and “Home monitoring of sleep with a temporary-tattoo EEG, EOG and EMG electrode array: a feasibility study.” by Shustak, S., Inzelberg, L., Steinberg, S., Rand, D., Pur, M. D., Hillel, I., . . . & Hanein, Y. (2019), Journal of neural engineering, 16(2), 026024)

### (3) Bittium

The electrode system is a product designed for application in medical emergency care and therefore not with the consideration that a person may apply it themselves. In the patent document cited below, however, further applications, such as sleep monitoring, are mentioned. However, there is no mention of the grid being applicable by the user themselves. Another person, such as, e.g., a trained nurse, is involved. (cf. https://shop.bittium.com/product/28/bittium-brainstatus-eeg-electrode-10pcs (21.7.2021) and US2015238106A)

The publication [V. Toral et al., “Cost-Effective Printed Electrodes Based on Emerging Materials Applied to Biosignal Acquisition,” in IEEE Access, vol. 8, pp. 127789-127800, 2020, doi: 10.1109/ACCESS.2020.3008945] introduces procedures with different materials for producing printed electrode systems, particularly for the application of EKG, EMG, and EOG.

Taking this into consideration, there is a need for a concept allowing for a self-applicable electrode arrangement and a better compromise between easy handling, high positional accuracy of the electrodes on a head of a user, good adhesion properties of the electrode arrangement on the head of the user, high signal quality and good wear comfort over several hours.

### SUMMARY

According to an embodiment, a device may have: an electrode arrangement with a plurality of electrodes for measuring electric signals on a face surface of a head of a user and behind an ear of the user, wherein the electrodes of the plurality of electrodes are wet electrodes; a carrier structure; wherein the carrier structure at least partially defines a relative position of the electrodes to one another; and wherein the carrier structure is shape-stable within a tolerance range without external force exerted by a user and deformable under external force exerted by the user, in order to change a distance between two adjacently arranged electrodes and to area-comprehensively adapt to face and head structures, wherein the carrier structure is configured to position at least a part of the electrode arrangement on the face surface and behind the ear; wherein the carrier structure is configured in the shape of a net structure with branching connecting ribs that follow a course of the face surface; and wherein the branched connecting ribs have a rib width corresponding to, at most, three times the electrode diameter of an electrode of the plurality of the electrodes.

According to another embodiment, a system may have: an inventive device as mentioned above; and an evaluation arrangement that is configured to evaluate the electric signals measured by the plurality of electrodes.

According to another embodiment, a method for using the inventive device as mentioned above may have the steps of: applying the device on a face surface of a head of a user by the user themselves or by another person; and monitoring the user by means of the electric signals measured by the plurality of electrodes.

According to a first aspect of the present invention, the invention have found that a problem arising from an autonomous application of an electrode arrangement results from the fact that, to date, the electrodes cannot be positioned correctly in a simple and efficient manner by the user on themselves. According to the first aspect, this difficulty is overcome in that a carrier structure is provided for the electrode arrangement, which connects the individual electrodes of the electrode arrangement to one another. Due to the carrier structure, distances between the electrodes and/or positions of the electrodes relative to one another are predefined. The carrier structure spans, e.g. a matrix, on which the electrodes of the electrode arrangement are arranged at predefined positions. Thereby, a simple, efficient, and precise positioning of the electrodes by the user on themselves is made possible as the carrier structure provides guidance. By affixing the carrier structure on a head or face of the user, the electrodes arranged at predefined positions on the carrier structure are automatically positioned correctly, within a tolerance range, on the head and/or on the face of the user. Due to the special design of the carrier structure, which connects the electrodes of the electrode arrangement to one another, a greater wear comfort and a good manageability are achieved. The device is particularly advantageous when compared to a placement of individual electrodes as this is accompanied by more effort regarding the installation and a lower security of reproducibility of the configuration of the electrodes than in the device presented herein. The proposed solution is intended, in particular, to remedy this and to allow for a quicker and simpler installation of the electrodes, particularly by the person themselves, so that no further (trained) person needs to be present.

Furthermore, the inventors have found that it is advantageous if the carrier structure has a flexibility that is dependent on whether the user exerts a force onto the carrier structure or not. This different flexibility allows for adapting a simple, efficient, and precise positioning of the electrodes on facial structures of different users.

Thus, when no force is exerted by the user, the carrier structure should be so dimensionally stable or shape-stable or rigid that, under the influence of gravity, it does not bend at all or only barely, or that it does not yield to gravity at all or only barely. The shape-stability may be achieved, e.g., in that the carrier structure has a material such as, e.g. polyurethane foam material and a certain thickness, e.g. in the range of 0.5 mm to 2 mm. Alternatively, the carrier structure may have a releasable stiffening element that is configured to stabilize the shape of the carrier structure. In that regard, the carrier structure may be reinforced, e.g. by means of the releasable stiffening element, for the process of applying the carrier structure by the user. In the following, this is referred to collectively as a carrier structure. The carrier structure span, for example, a matrix that, at a length of 10 cm, bends by a maximum of 15 cm, 10 cm, 8 cm, or 6 cm in the direction of gravity when oriented in a plane perpendicular to gravity. The carrier structure thus has a high degree of rigidity and/or a low resilience regarding gravity. This is based on the idea that due to the rigidity, the user may place carrier structure on their head and/or face in a simple and efficient manner without the structure collapsing, which would complicate the affixing. The shape of the carrier structure and/or the relative arrangement of the electrodes to one another with gravity acting on the carrier structure oriented perpendicular to gravity deviates from the shape of the carrier structure and/or the relative arrangement of the electrodes to one another with a force of zero Newton acting on the carrier structure (the shape of the carrier structure and/or the relative arrangement of the electrodes in a force equilibrium) by a maximum of 2 cm or a maximum of 3 cm. If, however, the carrier structure is oriented in parallel to gravity, the deviation amounts to a maximum of 2 cm or a maximum of 1 cm. This shape-stability of the carrier structure is relevant particularly as long as the device has not been affixed to a head surface of the user, i.e. on a face surface of the user and behind one ear of the user.

However, under external force exerted by the user, the carrier structure should be deformable and/or malleable, such that the carrier structure can be adapted depending on the facial structure of the user, so that irregularities, for example, formed by bone structures, may be adhered to in a planar manner and good adhesion is made possible. Moreover, such an electrode position of a first electrode on a face of a first user may be essentially aligned with an electrode position of the first electrode on a face of a second user. This ensures that, despite different head circumferences of users, the electrodes can be affixed to their faces at predetermined positions by the respective user within a tolerance range of up to 1 cm or up to 2 cm. The carrier structure is formed, such that, for example, a deformation in a range of, e.g., at least 2% to, e.g., a maximum of 10% to 20%, depending on the type of material, is possible. This is based on the idea that the carrier structure provides the user with assistance in positioning on the face and/or on the head of the user, and the carrier structure prescribes the positions of the electrodes on the face and/or on the head of a user due to the limited deformability, and the shape of the carrier structure can be adapted by the user only minimally to their specific facial structure. The carrier structure is thus configured, for example, to be adaptable to a head anatomy and/or face anatomy of the user, so that predefined positions of the electrodes on the carrier structure matches predefined points on the head and/or on the face of the user.

Accordingly, a device according to a first aspect of the present invention, comprises a carrier structure and an electrode arrangement having a plurality of electrodes for measuring electric signals on a face surface of a head of a user and optionally furthermore for measuring electric signals behind an ear of the user. The carrier structure defines, at least partially, a relative position of the electrodes to one another. The carrier structure is shape-stable within a tolerance range without external force exerted by a user and deformable under external force exerted by the user in order to change a distance between to adjacently arranged electrodes, i.e. two electrodes that are connected via a rib of the carrier structure, and/or to adapt to face and/or head structures (e.g. area-comprehensively, i.e. lying flat on the respective surface). A distance between two adjacently arranged electrodes is, e.g., adaptable by stretching, compressing, or twisting the rib of the carrier structure connecting the two electrodes. It is particularly advantageous that due to the deformability of the carrier structure, the user may adapt, for example, the carrier structure to curved parts on their face surface and/or head surface. Thereby, the carrier structure can be affixed flat on the respective surface, whereby, e.g., a robust adhesive bond between the carrier structure and the face and/or face surface is ensured. In other words, the carrier structure may be shape-stable within a tolerance range without external force exerted by a user, and deformable under external force exerted by the user in order to adapt area-comprehensively to face and/or head structures, so that the carrier structure lays with its entire area on the face surface and/or the head surface. Between the individual electrodes of the electrode arrangement, for example, the carrier structure is, for example, planar, in contact with the respective surface. The carrier structure is formed to position at least a part of the electrode arrangement on the face surface and optionally a further part of the carrier structure behind the ear of the user.

According to an embodiment, the electrode arrangement comprises wet electrodes, such as solid gel electrodes, for example. Thus, the electrodes of the plurality of electrodes are wet electrodes. In other words, the electrode arrangement advantageously comprises a plurality of wet electrodes. Wet electrodes offer a higher signal quality than dry electrodes as highly viscous conductive gel permeates the skin better and thus reduces the impedance. When using wet electrodes, the gel, e.g. conductive gel or solid gel, is already pre-applied, e.g. as a conductive gel lens or a solid gel lens. The pre-applied conductive gel or solid gel is protected, for example, by a removable protective element, such as a protective film. Due to the pre-application of the gel, the user does not have to apply it themselves, and the application of the device by the user is facilitated. Furthermore, the inventors have found that due to the use of wet electrodes, the wear comfort can be improved compared to dry electrodes as there is no need to apply pressure for connecting the electrode and the skin, and a significantly lower electrode weight is achieved. When using dry electrodes, these have to be pressed onto the skin with a force of at least 1 N to 4 N (1 Newton to 4 Newton) or more in order to be able to measure signals with an impedance in the range of 250 to 50 kOhm. With wet electrodes, however, the connection between the respective electrode and the skin is achieved by an electrolyte gel, e.g. a solid gel, whereby the carrier structure can be formed to press the electrodes onto the skin without force or with only slight force. This means that a force of a maximum of 0.2 N is sufficient to record signals with an impedance of below 50 kOhm or much less. This small force may already be achieved, for example, by sticking the carrier structure with the electrode arrangement to the face surface of the user. Thus, wet electrodes allow for recording signals at a better signal-noise ratio than dry electrodes. In a further advantageous embodiment, the electrode surface of an electrode of the plurality of electrodes should have a diameter of less than 1 cm in order to even be able to place the plurality of electrode in the face and on the head. This is advantageous for long-term applications spanning several hours, overnight or multiple days.

According to an embodiment, the carrier structure follows the face surface as a human face shape, and the plurality of electrodes are arranged on the carrier structure. The carrier structure follow, e.g., the face surface in the sense that the carrier structure spans a matrix that follows distinctive facial lines. Distinctive facial lines are, e.g., the eyebrow, the cheek bone (i.e. the zygomatic bone), the rounding behind the ear and the mandible. The carrier structure follows, e.g., the face surface in the sense that the carrier structure spans a matrix that follows the shape of the eyebrow of the user and follows the shape of the ear behind the ear of the user. The carrier structure is configured, e.g., to position the plurality of electrodes at distinctive points on the face surface of the user. An upper cheekbone, a point in front of the tragus of the ear, a point on the forehead vertically above the nasion, the chin and/or the mastoid behind the ear are distinctive points, for example. A first electrode position assigned to a first electrode of the plurality of electrodes corresponds, e.g., to a position of a first distinctive point on the face surface; and a second electrode position assigned to a second electrode of the plurality of electrodes corresponds, e.g., to a position of a second distinctive point on the face surface; and a third electrode position assigned to a third electrode of the plurality of electrodes corresponds results from the first electrode position and the second electrode position on the face surface, e.g., at a predetermined position within a tolerance range determined by the carrier structure. The first electrode position and the second electrode position represent, for example, reference positions for positioning further electrodes of the plurality of electrodes. The tolerance range results, for example as explained above, from the deformability of the carrier structure when a user exerts force. Due to the deformability by the user, a tolerance range of a maximum of 2 cm or a maximum of 1 cm around a position on a face surface of a third electrode is achieved. The carrier structure is configured, e.g., to position the plurality of electrodes on a corresponding plurality of predetermined positions, e.g., the distinctive points, on the head surface of a user, i.e. on the face surface and/or behind the ear. The tolerance range defines, e.g., an area of a maximum of 2 cm or of a maximum of 1 cm around a predetermined position of the respective electrode on the head surface. The tolerance range indicates, e.g., the reproducibility of how the carrier structure the carrier structure positions the electrodes of the electrode arrangement on different heads, e.g., with different head circumferences, on distinctive points and/or the accuracy of hitting these distinctive points. The electrode arrangement does not have to be positioned even more accurately. Within this tolerance range, a high signal quality is achieved. The carrier structure is configured, e.g., to connect the third electrode to the first and second electrodes via a connecting rib, wherein a shape and/or a length of the connecting rib is dependent on the first and the second electrode position. Optionally, the shape and/or the length of the connecting rib further depends on the face shape. Thus, the shape of the carrier structure takes into account that the connecting rib does not pass over an eye, an ear, a nose, or a mouth of a user. The specially shaped carrier structure and the special arrangement of the plurality of electrodes on the carrier structure allow for the ability to arrange the electrodes on the face of the user with a high wear comfort and high degree of precision. Due to the precision in positioning, a high signal quality of positions on the head defined specifically for the application case.

According to an embodiment, the first distinctive point of the face surface corresponds to an upper cheekbone, and the second distinctive point corresponds to a position in front of the tragus of an ear of the user. The selection of these two points as reference positions for position further electrodes, such as the third electrode, is based on that these two points are easy to feel on the face surface and can thus be located exactly by the user. The inventors recognized that by means of these two points, the carrier structure with the electrode arrangement may be oriented very precisely on the face surface of the user. Because the reference positions (i.e. the first electrode position and the second electrode position) correspond to characteristic points in the face of the user, e.g., electrodes of the plurality of electrodes arranged relative to the reference points on the carrier structure due to the shape of the carrier structure can be positioned easily on predetermined positions on the face surface. The remaining electrodes (e.g. the fourth electrode to the nth electrode, wherein the plurality of electrodes comprises n electrodes) of the plurality of electrodes are, e.g., arranged on the carrier structure relative to the first electrode and the second electrode. Thereby, the electrode arrangement can be affixed to the user's face surface precisely in a simple and efficient manner by the user, whereby a high signal quality is achieved.

According to an embodiment, the carrier structure includes a connecting rib (e.g. a first connecting rib) that at least partially connects the first electrode position and/or the second electrode position, on one side, to the third electrode position, on the other side, and wherein the third electrode position matches, within a tolerance range of 1 to 2 cm determined by the carrier structure, a position on a forehead of the face surface in an area of 1 cm to 3 cm above the eyebrow line, vertically above a nasion of the user. The third electrode position should therefore correspond to a position in the area of 1 cm to 3 cm above the eyebrow line, vertically above the nasion of the user. The inventors, however, recognized that a high signal quality may likewise be achieved if the third electrode position is located within a tolerance range of 1 to 2 cm on both sides vertically relative to the area. The tolerance range runs vertically to the area vertically above a nasion and/or vertical relative to an extension line of the tip of the nose to the nasion and measures, e.g. 1 cm or a maximum of 2 cm. An optimal signal quality is achieved if the third electrode position is arranged 2 cm above the eyebrow line, vertically above the nasion of the user. Due to the connecting rib of the carrier structure, the third electrode position is prescribed, whereby the user is able to affix the third electrode very precisely on their face surface. In doing so, the connecting rib is designed to allow a certain tolerance range, however, it simultaneously avoids that the user affixed the electrode outside of a predetermined area on the face surface. This ensures that the user can attach the third electrode of the electrode arrangement easily as the connecting rib of the carrier structure ensures that the third electrode is arranged on the face surface such that a good signal quality from a specifically defined location is achieved for the electric signal measured using the third electrode.

According to an embodiment, the carrier structure includes a connecting rib (e.g. a third connecting rib) that at least partially connects the first electrode position and/or the second electrode position, on one side, to a fourth electrode position that is assigned to a fourth electrode of the plurality of electrodes, on the other side, and wherein the fourth electrode position matches, within a tolerance range determined by the carrier structure, a position behind the ear, such as, e.g., a predetermined position such as a mastoid of the user. The tolerance range defines, for example, an area of a maximum of 1 cm or a maximum of 2 cm around a prescribed and/or predetermined position behind the ear. Optionally, further electrodes of the plurality of electrodes, e.g. a sixth, a seventh, an eighth, and a ninth electrode, are arranged on the connecting rib. The positions of the further electrodes match a corresponding number of positions behind the ear of the user within the tolerance range determined by the carrier structure. The sixth, seventh, eighth, and ninth electrodes are arranged, e.g., on the connecting rib relative to the fourth electrode position. A shape and/or length of the connecting rib depends on the first and second electrode positions, for example. The connecting rib is designed, e.g., to define the fourth electrode position relative to the first and/or second electrode position. The connecting rib leads, for example, from a face of the user above the ear of the user to behind the ear, wherein behind the ear, the connecting rib follows the shape of the ear. Optionally, the third connecting rib and the first connecting rib comprise a common segment. The common segment branches off, e.g., to the first connecting rib and the third connecting rib. At the point of branching, a further electrode of the plurality of electrodes is arranged, for example.

According to an embodiment, the carrier structure includes a connecting rib (e.g. a second connecting rib) that at least partially connects the first electrode position and/or the second electrode position, on one side, to a fifth electrode position that is assigned to a fifth electrode of the plurality of electrodes, on the other side, and wherein the fifth electrode position matches, within a tolerance range determined by the carrier structure, a position, e.g., a predetermined position on a chin of the face surface of the user. The tolerance range defines, for example, an area of a maximum of 1 cm or a maximum of 2 cm around a prescribed and/or predetermined position on the chin. The connecting rib is designed to be deformed by the user in order to be able to adapt the fifth electrode position to the face structure of the user, wherein it is ensured that the fifth electrode position corresponds to a position within the tolerance range around the chin of the user. A shape and/or length of the connecting rib depends on the first and second electrode positions, for example. The connecting rib is designed, e.g., to define the fifth electrode position relative to the first and/or second electrode position.

According to an embodiment, the carrier structure comprises an adhesive material for affixing the plurality of electrodes and the carrier structure on the face surface. Optionally, the adhesive material is covered by a removable protective film.

According to an embodiment, the plurality of electrodes further comprise an adhesive electrolyte gel for affixing the plurality of electrodes and the carrier structure on the face surface. The adhesive electrolyte gel is covered at least in some areas by a removable protective film. The electrodes with the adhesive electrolyte gel and the adhesive material may comprise, e.g., a common removable protective film or separate protective films. The electrodes with the adhesive electrolyte gel are so-called wet electrodes. The adhesive material is arranged, e.g., on the side of the carrier structure on which the electrode arrangement is arranged as well.

According to an embodiment, the protective film covering the adhesive material and optionally furthermore the adhesive electrolyte gel comprises a plurality of adhesive film portions that can be removed from the carrier structure and the plurality of electrodes independently of one another. This allows the user to affix individual portions of the carrier structure and the electrode arrangement on their face one after the other in a simple and efficient manner as this avoids that parts of the carrier structure that are applied at a later point in time stick together. Optionally, a first protective film portion of the plurality of protective film portions covers a first subset of the plurality of electrodes and of the carrier structure, and a second protective film portion of the plurality of protective film portions covers a disjoint second subset of the plurality of electrodes and of the carrier structure. The first protective film portion covers, e.g., a first section of the carrier structure, wherein the first section and the second section do not overlap. The first subset of the plurality of electrodes is arranged, e.g. in the first section of the carrier structure, and the second subset of the plurality of electrodes is arranged, e.g. in the second section of the carrier structure. Optionally, certain subsets of the carrier structure may also be designed to not be adhesive, for example for the path behind the ear. Thus, for example, a region of the carrier structure located on the side on which the electrode arrangement is arranged may not comprise adhesive material.

According to an embodiment, the carrier structure is formed in the shape of a net structure, e.g., a matrix, with branching connecting ribs that follow the course of the face surface. The branching connecting ribs follow, e.g., distinctive lines and/or shapes of the human face shape. The branching connecting ribs follow, e.g., the course of the face surface in the sense that the connecting ribs follow the shape of the eyebrow of the user, e.g., along the eyebrow and along the cheekbone and around the eye, and follow the shape of the ear behind the ear of the user. Optionally, the connecting ribs may furthermore run along the mandible towards the chin of the user. The fact that the connecting ribs follow the course of the face surface and are deformable by force results in a high wear comfort for the user.

According to an embodiment, the branching connecting ribs have a rib width corresponding to, at most, 1.5 times, two times or three times the electrode diameter of an electrode of the plurality of the electrodes. Due to the narrow design of the connecting ribs, the wear comfort is further increased as only a small part of the face surface is in contact with the carrier structure. Due to the increased wear comfort, the device may be used on the user for a longer period, for example overnight. Furthermore, the inventors recognized that the narrow connecting ribs facilitate adapting to user-dependent face shapes.

According to an embodiment, one connecting rib of the branching connecting ribs has a curved end so that the carrier structure has a curved end piece that is formed to be affixed behind an ear of the user, and wherein the connecting rib is formed to run from the face of the user, above the ear, to the curved end piece. At least one electrode of the plurality of electrodes is arranged at the curved end piece. The inventors recognized that the course of the connecting piece above the ear facilitates the autonomous affixing of the device on the face surface of the user as the connecting rib is formed to be placed on the upper base of the ear, for example, during the application in order to facilitate the correct orientation. The device is supported on the ear by means of the connecting rib, which allows for an efficient arrangement and affixing of the remaining branching connecting ribs on the face surface as the connecting rib towards the ear of the user does not have to be supported by a hand. The curved end piece is based on the idea that the user may hook it behind the ear and thus, stabilizes the device upon autonomous affixing on the face surface of the user and/or already orients the device at least partially in the correct target position on the face surface. Thus, the connecting rib enables an autonomous affixing of the device by the user and reduces a time consumption for the application.

In the area of the connecting rib running above the ear, for example, no adhesive material is applied, or a non-removable protective film is arranged covering the applied adhesive material in that area. Thereby, the device is prevented from bonding with hair, whereby the wear comfort is optimized. The inventors recognized that the connecting rib may be placed firmly on the upper base of the ear, whereby the autonomous affixing of the device on the face surface of the user is facilitated.

According to an embodiment, the carrier structure has an ear are that is formed to run above an ear of the user. In other words, a part of the carrier structure runs above the ears of the user. This part running above the ear may be considered an ear area. The inventors recognized that a course of the carrier structure above the ear facilitates the autonomous affixing of the device on the face surface of the user as the ear area is formed to be place, e.g., on the upper base of the ear of the user. The device is supported on the ear via the ear area, which enables an efficient arrangement and affixing of the remaining carrier structure on the face surface.

According to an embodiment, the ear area of the carrier structure is part of a connecting rib that, originating from at least a first electrode position that is assigned to a first electrode of the plurality of electrodes and/or a second electrode position that is assigned to a second electrode of the plurality of electrodes, specifies a fourth electrode position that is assigned to a fourth electrode of the plurality of electrodes. The fourth electrode position corresponds to a position behind the ear of the user. Optionally, the fourth electrode constitutes a reference electrode (REF) or a ground electrode for the plurality of electrodes. As an alternative thereto, e.g. the fourth electrode may represent a reference electrode (REF), and an electrode arranged adjacently to this electrode, e.g., the electrode 110₅, may represent a ground electrode (GND).

According to an embodiment, the carrier structure or a shape of the carrier structure is configured to position the plurality of electrodes at a corresponding plurality of positions on the face surface. The special shaping of the carrier structure defines, e.g., the positions of the electrodes arranged on the carrier structure on the face surface. The plurality of positions is configured for a detection of the electric signals on the face surface for at least a linear combination of the electric signals for mapping signals from a predetermined head region of the user. The shape of the carrier structure as well as, e.g., the positioning of the electrodes of the electrode arrangement on the carrier structure enables a detection of electric signals from different head regions of the user, such as different brain and muscle regions. The arrangement of the electrodes on the carrier structure is designed to derive, e.g. based on linear combinations of the electric signals measured by the electrodes, an electroencephalogram for different brain regions [e.g., in a prefrontal region (i.e. in a front region of the frontal lobe (Fp)), frontal region (i.e. in the front or in the frontal lobe (F)), in the temple region (e.g. temporal (T)), parietal (P), in a region of the back of the head (i.e. occipital (O)), and in a central brain region (i.e. central (C))], to derive an electrooculogram (vertical, horizontal, and diagonal), and/or to derive an electromyogram, particularly in the area of the chin, for example. Due to the shape of the carrier structure being such that the electrodes arranged on the carrier structure correspond to predetermined positions on the face surface of the user, the linear combinations of the measured signals also allow deriving head regions and/or brain and muscle regions of the user where no electrode directly measures a signal. This allows minimizing the number of electrodes and thus reducing costs, but also increasing the wear comfort for the user as fewer areas of the face region are covered by the device, only hairless skin regions are used if possible, and the low number additionally reduces the weight of the device. Finally, information on heart activity can also be gathered from the detected signal so that an electrocardiogram (EKG) can also be derived from the data using an appropriate signal analysis.

According to an embodiment, the device has a signal output that is configured to provide an output signal based on the electric signals measured by the plurality of electrodes. The signal output is coupled to the electrodes of the electrode arrangement of the device, e.g., via electric lines. Optionally, the device further comprises a signal amplifier that is configured to receive the measured electric signals from the signal output. The device comprises, e.g., fastening means defining a position of the signal amplifier on a back of the head, an arm, a shoulder, a chest, or a neck of the user.

According to an embodiment, the fastening means is a collar or scarf that is configured to affix the signal amplifier on the neck of the user, below a bony projection, the mastoid process, and between the two muscles, the sternocleidomastoid muscle and the trapezius. This is based on the knowledge that this position of the signal amplifier results in a high wear comfort of the user. In this position, sleeping with the device is also comfortable. Thus, this positioning of the signal amplifier improves a use of the device over several hours, for example overnight, or even over several days for performing a long-term screening.

According to an embodiment, the carrier structure comprises polyurethane material (e.g. polyurethane film material), polymer material, and/or silicone material as the carrier material. The carrier material is stretchable or elastic and, e.g., non-rigid. The non-rigidity allows for a planar adaptation to any unevenness of face and head structures. If the structure (e.g., the carrier structure with a maximum thickness of 0.1 mm, 0.3 mm, or 0.5 mm, such as 0.2 mm, for example) is placed on a radial elevation (assumed herein as a cylinder) with a diameter of, e.g., 0.5 cm and a height of 1 cm, the carrier material may simultaneously come to lie, either following gravity due to its own weight or due to minimal force application (<1 N), on the circle surface and on four locations of the lateral surface of the cylinder, of which two of the areas on the lateral surface are opposite one another and the connecting lines of the respectively opposite surfaces are perpendicular to one another, without the carrier structure being damaged (tearing or breakage). Here, the structure follows the 90° corner of the area of the circle surface to the lateral surface of the cylinder with a tightly fitting radius of a maximum of 1 mm. Non-rigidity may be understood to mean, e.g., that a structure is deformable in a thickness direction of the structure simultaneously along two lateral directions perpendicular to one another without being damaged and/or that the structure can be twisted multiple times without being damaged, i.e. when a torsional moment acts on the structure in order to twist the same multiple times about an axis perpendicular to the thickness direction.

According to an embodiment, the carrier structure comprises polyurethane material (e.g. polyurethane film material), polymer material, and/or silicone material as the carrier material. The carrier material is stretchable or elastic and, e.g., non-rigid. The electrodes are connected to traces. The traces comprise a slung course on or in the carrier structure in order to stretch correspondingly when the carrier structure is stretched. The traces having the slung course, such as wavelike, a meandering or a jagged shape, enables the device to be adaptable to face shapes of users that are different from one another, as the traces do not tear upon deformation of the carrier structure due to external application of force by the user. The traces are, e.g. integrated into the carrier material, or arranged on a surface of the carrier structure. The traces may be produced on or in the carrier structure by means of methods that are also used in the production of circuit boards. Thus, the traces may be printed on or etched on.

According to an embodiment, the carrier structure is configured to extend a distance between two electrodes of the electrode arrangement by a maximum of 20%, 15%, 10%, or 5%. The distance is a distance between two adjacent electrodes between which no further electrode is arranged on the carrier structure. The extension is effected, for example, by external force exerted on the carrier structure by the user. The responsible factors for the distance being extendable by a maximum of 20%, 15%, 10%, or 5% are, inter alia, the stretchable or elastic carrier material and the slung traces. It is advantageous if the carrier structure is configured to extend the distance between two electrodes, e.g., by at least 1%, 3%, or 5%. It is particularly advantageous if the carrier structure is configured to extend the distance between two electrodes, e.g., in a range of 3% to 20%, or 1% to 5%. Due to the minimal requirement for the carrier structure to be configured to be able to extend the distance by at least 1%, 3%, or 5%, it is achieved that the device is accompanied by great wear comfort for the user, adapts well to any unevenness, and is suitable for different face shapes. Due to the maximum limit that the carrier structure should be configured to extend the distance by a maximum of 20%, 15%, 10%, or 5%, the user is prevented from positioning the electrodes of the electrode arrangement arranged on the carrier structure at positions on the face surface that deviate, due to external force application, from the electrode positions on the face surface prescribed by the carrier structure by more than a certain tolerance range. Regarding the tolerance range, reference is made to the above explanations. The possible extension of the distance is limited, inter alia, by the clearance generated in the traces due to the slung course in or on the carrier structure and due to the stretchability and/or elasticity of the carrier material.

According to an embodiment, the carrier structure comprises polyurethane foam and optionally, in addition, a polyurethane film (e.g. as a stiffening element) as a carrier material. The carrier material is stretchable or elastic and e.g. non-rigid. The non-rigidity allows for a planar adaptation to any unevenness of face and head structures. If the structure (e.g., the carrier structure with a thickness in a range of 0.5 mm to 2 mm) is placed on a radial elevation (assumed herein as a cylinder) with a diameter of, e.g., 0.5 cm and a height of 1 cm, the carrier material may simultaneously come to lie, due to minimal force application (<1 N), on the circle surface and on four locations of the lateral surface of the cylinder, of which two of the areas on the lateral surface are opposite one another and the connecting lines of the respectively opposite surfaces are perpendicular to one another, without the carrier structure being damaged (tearing or breakage). Here, the structure follows the 90° corner of the area of the circle surface to the lateral surface of the cylinder with a tightly fitting radius of a maximum of 1 mm. Non-rigidity may be understood to mean, e.g., that a structure is deformable in a thickness direction of the structure simultaneously along two lateral directions perpendicular to one another without being damaged and/or that the structure can be twisted multiple times without being damaged, i.e. when a torsional moment acts on the structure in order to twist the same multiple times about an axis perpendicular to the thickness direction.

According to an embodiment, the carrier structure comprises polyurethane foam and optionally, in addition, a polyurethane film (e.g. as a stiffening element) as a carrier material. The carrier material is stretchable or elastic and e.g. non-rigid. The electrodes are connected to cables. The cables are guided, having a clearance, along a surface of the carrier structure facing away from the electrode arrangement. The clearance limits a force transmission of a stretching of the carrier structure to the cables. The clearance corresponds to, for example, a wound or loose course of the cables on the carrier structure. The carrier structure may comprise, e.g., clamping points spaced apart from one another, which are configured to affix the cables to the carrier structure, i.e. clamp them on, wherein between the clamping points, the cables are not affixed to the carrier structure and have a greater length than the distance between the respective clamping points. Alternatively, the carrier structure may comprise, e.g., guide rings that are spaced apart from one another, which are configured to guide the cables along the carrier structure, wherein the cables run loosely through the guide rings, and the cables have a greater length between the guide rings than the distance between the respective guide rings. Similar to what has been described above regarding the traces, the elastic carrier material as well as the clearance in the guiding of the cables along the carrier structure, allow the device to be adapted to face shapes of users that are different from one another as the cables do not tear upon a deformation of the carrier structure by external force exerted by the user.

According to an embodiment, the carrier structure is configured to extend a distance between two electrodes of the electrode arrangement by a maximum of 20%, 15%, or 10%. The distance is a distance between two adjacent electrodes between which no further electrode is arranged on the carrier structure. The extension is effected, for example, by external force exerted on the carrier structure by the user. The responsible factors for the distance being extendable by a maximum of 20%, 15%, or 10% are, inter alia, the stretchable or elastic carrier material and the clearance in guiding the cable along the carrier structure. It is advantageous if the carrier structure is configured to extend the distance between two electrodes, e.g., by at least 1%, 3%, or 5%. It is particularly advantageous if the carrier structure is configured to extend the distance between two electrodes, e.g., in a range of 3% to 15%, or 5% to 20%. Due to the minimal requirement for the carrier structure to be configured to be able to extend the distance by at least 1%, 3%, or 5%, it is achieved that the wear comfort for the user is high and that the device is suitable for users with different face shapes. Due to the maximum limit that the carrier structure should be configured to extend the distance by a maximum of 20%, 15%, or 10%, the user is prevented from positioning the electrodes of the electrode arrangement arranged on the carrier structure at positions on the face surface that deviate, due to external force application, from the electrode positions on the face surface prescribed by the carrier structure by more than a certain tolerance range. Regarding the tolerance range, reference is made to the above explanations. The possible extension of the distance is limited, inter alia, by the clearance in the guiding of cables along the carrier structure and due to the stretchability and/or elasticity of the carrier material.

According to an embodiment, the carrier structure is adapted for different head circumferences by means of the stretchable or elastic carrier material and, e.g., by means of the special guiding of the traces or cables in, on or along the carrier structure, wherein the different head circumferences vary by a maximum of 5 cm. According to an alternative embodiment, the carrier structure is adapted, by means of the elastic carrier material and, e.g., by means of the special guiding of the traces or cables in, on or along the carrier structure, for positioning the at least one part of the electrode arrangement on the face surface of the head of the user having a head circumference in a range of 50 cm to 55 cm, or 55 cm to 60 cm, or 60 cm to 65 cm.

According to an embodiment, the carrier structure comprises a removable stiffening element on a side facing away from the electrode arrangement, which stiffening element is configured to stiffen the carrier structure at least locally. The stiffening element may comprise, e.g., paper material, polyurethane foam, or polyimide material. With the aid of the stiffening element, an autonomous positioning of the device on the face surface of the user is facilitated as the stiffening element prevents the carrier structure from changing its shape under the influence of gravity. By preventing the shape of the carrier structure from changing, the carrier structure is configured to guide the user as to which positions on their face surface the electrodes of the electrode arrangement should be affixed to, as the shape of the carrier structure defines the electrode positions of the electrodes on the face surface. A further advantage of the stiffening element is that it prevent the carrier structure from bending such that different parts of the carrier structure stick together, thus limiting the functionality of the device. The stiffening element is removable so that it may be removed after affixing the structure and the property of non-rigidity and elasticity of the carrier structure for a planar adaptation of the carrier structure to the face is fully ensured.

According to an embodiment, the stiffening element is configured to maintain a deformation of the carrier structure caused by external force exerted by the user when the external force exerted by the user is over. Thereby, the wear comfort is increased. The wear comfort is increased particularly when the stiffening element is arranged, e.g., on the carrier structure in an area corresponding to an area around an eye of the user or a cheek area of the user. In the chin region, e.g., no stiffening element should be arranged as the wear comfort for the mouth movement of the user, in particular, if the carrier structure is configured to be reversibly elastic or slightly stretchable in this area.

According to an embodiment, the carrier structure comprises at least one extension at a position at which an electrode of the electrode arrangement is arranged. The extension is, e.g., a bulging of the carrier structure, a branching part of the carrier structure with a maximum length of 2 cm, 1.5 cm, or 1 cm, or an extension of the carrier structure with a maximum length of 2 cm, 1.5 cm, or 1 cm. The carrier structure runs and/or branches, e.g., within one plane, and an extension extends laterally out of said plane. The extension does not run orthogonally to the plane. For example, the surface of the carrier structure, on which the electrode is arranged, defines a plane, and the extension runs within said plane. The extension runs, for example, orthogonally to a normal vector of the carrier structure at a position at which an electrode of the electrode arrangement is arranged. Optionally, adhesive material is arranged on the extension for improving the adhesiveness of the electrodes on the face surface of the user. Without adhesive material, the extension facilitates a handling of the device, particularly an autonomous application and removal of the electrode to/from the face surface of the user. One the one hand, the extension makes the electrode is easier to hold on to without touching the electrode and/or the sensitive sensor unit of the electrode, and, on the other hand, the extension stabilizes the electrode on the face surface when the same is being positioned by the user.

According to an embodiment, the plurality of electrodes comprises active electrodes having a circuit that is configured to perform an impedance conversion of the measured electric signal, and/or to amplify the measured electric signal. Due to the electrode arrangement comprising active electrodes, the signals is of higher quality regarding the signal-noise ratio and is less prone to signal disturbances due to movements of the electrode.

As an alternative or in addition to the active electrodes, the plurality of electrodes may comprise passive electrodes. If the electrode arrangement comprises passive electrodes, e.g., an impedance converter and/or a signal amplifier is used in most cases, which should be arranged on the body of the user, close to the electrode arrangement of the device. Optionally, the device may comprise an impedance convertor and/or signal amplifier that is coupled with a signal output of the device. The use of passive electrodes entails the advantage that, compared to using active electrodes, a weight of the carrier structure with the electrode arrangement arranged thereon may be reduced. This increases the wear comfort on the face surface of the user.

A further embodiment is directed at a device comprising a carrier structure and an electrode arrangement having a plurality of electrodes for measuring electric signals on a face surface of a head of a user and behind an ear of the user. The electrodes of the plurality of electrodes are wet electrodes (with gel or solid gel). The carrier structure defines at least partially a relative position of the electrodes to one another, and the carrier structure is configured to be in planar contact with the face surface of the user between the individual electrodes of the electrode arrangement. Thus, the special design of the carrier structure makes it possible for it to lie flat on the face surface and to follow face and head structures and/or adapts to the same. Furthermore, the carrier structure is configured to position at least a part of the electrode arrangement on the face surface and behind the ear. The electrodes are connected either to traces having a slung course on or in the carrier structure, or to cables being guided, with a clearance, along on a surface of the carrier structure facing away from the electrode arrangement. Due to the slung course of the traces, or e.g., due to the clearance in the guiding of the cables, the carrier structure is configured to stretch without the traces or the cables being damaged as the traces or cables can run in correspondence with the stretching of the carrier structure. The clearance with which the cables are guided along the carrier structure limit, e.g. a force transmission of a stretching of the carrier structure to the cables. The device is based on the same considerations as the device described above. The device may be supplemented with all features and functions that are also described regarding the device described above.

A further embodiment is directed at a device comprising a carrier structure and an electrode arrangement having a plurality of electrodes for measuring electric signals on a face surface of a head of a user and behind an ear of the user. The electrode arrangement is arranged, e.g., on a first side and/or first surface of the carrier structure. The electrodes of the plurality of electrodes are wet electrodes (with gel or solid gel). The carrier structure defines at least partially a relative position of the electrodes to one another. The carrier structure comprises a carrier substrate of polyurethane film material and a stiffening element arranged on the carrier substrate in a removable manner. The first side and/or first surface of the carrier structure on which the electrode arrangement is arranged is located, e.g., opposite the stiffening element arranged on the carrier substrate in a removable manner. In other words, the electrode arrangement, the carrier substrate, and the stiffening element arranged on the carrier substrate in a removable manner are arranged, for example, in this order. The carrier structure with the stiffening element arranged on the carrier substrate in a removable manner is formed to be shape-stable within a tolerance range, and the carrier structure without the stiffening element arranged on the carrier substrate in a removable manner is formed to be shape-instable and/or non-rigid. The carrier structure is configured to position at least a part of the electrode arrangement on the face surface and behind the ear. The device is based on the same considerations as the device described above. The device may be supplemented with all features and functions that are also described regarding the device described above.

According to an embodiment, the carrier substrate can be reversibly twisted in itself multiple times and/or be reversibly deformed in a thickness direction of the carrier structure simultaneously along two lateral directions perpendicular to one another, in order to form the carrier structure to be shape-instable without the stiffening element removably arranged on the carrier substrate. In other words, the carrier structure is shape-instable without the stiffening element removably arranged on the carrier substrate, such that the carrier substrate, within the scope of an intended use, can be twisted in itself multiple times and/or the carrier substrate can be deformed in a thickness direction of the carrier structure simultaneously along two lateral directions perpendicular to one another, within the scope of an intended use. The deformations and twists of the carrier substrate can occur without damage (tears or breakage). It should be noted that at a point at which the carrier structure is deformed or twisted, a normal vector, which is orthogonal to a surface of the carrier structure facing away from the electrode arrangement, defines the thickness direction. The two lateral directions perpendicular to one another (i.e. a first lateral direction and a second lateral direction) span a plane perpendicular to the normal vector. Thus, the carrier substrate is configured to, for example, simultaneously deform and/or bend a surface of the carrier substrate located in the first lateral direction and a surface of the carrier substrate located in the second lateral direction upwards or downwards, as viewed from a deformation point, i.e. in a thickness direction. This type of deformation may occur without external force application, following gravity. If traces, e.g. having a slung course, are arranged in or on the carrier substrate, the type of deformation described above may occur under minimal force application (<1 N). The twisting of the carrier substrate multiple times as described above, is a torsion about an axis along the first lateral direction, the second lateral direction, or also a different direction within the plane spanned by the two lateral directions that are perpendicular to one another. Shape-instability and/or non-rigidity is understood to mean that all deformations and/or twists described above can occur without damaging the carrier substrate.

According to an embodiment, the carrier substrate is deformable in the thickness direction simultaneously along the two lateral directions that are perpendicular to one another, with a bending radius in a range of 0.1 mm to 1 mm. The carrier substrate is so shape-instable that the carrier substrate itself can adapt to any minor unevenness, for example wrinkles. Due to the shape-instability, it is achieved that the carrier structure can lie flat on the face surface of the user and even in the case of an unevenness, a projection of the carrier structure can be reduced or even completely avoided. Thereby, a better attachment of the device to the face surface of the user is achieved as a very great adhesive surface of the carrier structure can be brought into contact with the face surface of the user. Furthermore, the wear comfort is increased and long-term measurements are made possible.

The shape-instability described above is achieved, for example, by means of a maximum thickness of 0.5 mm of the carrier substrate. The thickness corresponds to an expansion of the carrier substrate in the thickness direction.

Without external force exerted by a user, the carrier structure is thus shape-stable within a tolerance range if the removable stiffening element is arranged on the carrier substrate. Furthermore, the carrier structure is deformable under external force exerted by the user in order to change a distance between two adjacently arranged electrodes and to area-comprehensively adapt to face and head structures when the stiffening element is removed from the carrier substrate.

A further embodiment is directed at a device comprising a carrier structure and an electrode arrangement with a plurality of electrodes for measuring electric signals on a face surface of a head of a user and behind an ear of the user. The electrodes of the plurality of electrodes are wet electrodes (with gel or solid gel). The carrier structure defines at least partially a relative position of the electrodes to one another and comprises polyurethane foam as the carrier material. The carrier structure is shape-stable within a tolerance range without external force exerted by a user and is deformable under external force exerted by the user in order to adapt to face and head structures in an area-comprehensive manner. The carrier structure is configured to be reversibly twisted in itself multiple times and/or to be reversibly deformed in a thickness direction of the carrier structure simultaneously along two lateral direction perpendicular to one another. The carrier structure is configured to position at least a part of the electrode arrangement on the face surface and behind the ear. The device is based on the same considerations as the device described above. The device may be supplemented with all features and functions that are also described regarding the device described above.

In other words, under the external force exerted by the user, the carrier substrate can be twisted, within the scope of an intended use, in itself multiple times and/or be deformed in a thickness direction of the carrier structure simultaneously along two lateral directions perpendicular to one another, within the scope of an intended use. The deformations and twists of the carrier substrate can occur without damage (tears or breakage). It should be noted that at a point at which the carrier structure is deformed or twisted, a normal vector, which is orthogonal to a surface of the carrier structure facing away from the electrode arrangement, defines the thickness direction. The two lateral directions perpendicular to one another (i.e. a first lateral direction and a second lateral direction) span a plane perpendicular to the normal vector. Thus, the carrier structure is, for example, configured to be deformed by a user under external force application such that, as viewed from a deformation point, a surface of the carrier substrate located in the first lateral direction and a surface of the carrier substrate located in the second lateral direction can be simultaneously deformed and/or bent upwards or downwards i.e. in a thickness direction. The twisting of the carrier substrate multiple times as described above, is a torsion about an axis along the first lateral direction, the second lateral direction, or also a different direction within the plane spanned by the two lateral directions that are perpendicular to one another, and/or a torsion about an axis arranged in a plane perpendicular to the thickness direction. All deformations and/or twists described above can occur without damaging the carrier substrate.

According to an embodiment, the carrier substrate is deformable in the thickness direction simultaneously along the two lateral directions that are perpendicular to one another, with a bending radius in a range of 0.1 mm to 1 mm. Due to the shape-instability, it is achieved that the carrier structure can lie flat on the face surface of the user and even in the case of an unevenness, a projection of the carrier structure can be reduced or even completely avoided. Thereby, a better attachment of the device to the face surface of the user is achieved as a very great adhesive surface of the carrier structure can be brought into contact with the face surface of the user. Furthermore, the wear comfort is increased and long-term measurements are made possible.

The carrier structure comprises a thickness, e.g., in the thickness direction, in a range of 0.5 mm to 2 mm. A thickness in this range allows for a shape-stability without external force application and a shape-instability under external force application.

A further embodiment relates to a system with a device according to one of the embodiments described above and having an evaluation arrangement. The evaluation arrangement is configured to evaluate the electric signals measured by the plurality of electrodes.

According to an embodiment, the evaluation arrangement is configured to provide an information on a sleep architecture of the user based on the electric signals; or to provide an information on epileptic behavior of the user; or to indicate a neurological disorder of the user; or to provide a cognitive psychological information. Thus, the system according to the invention allows for a domestic or outpatient monitoring for patients with epilepsy or suspected epilepsy, or for patients with other neurological disorders or suspected disorders (e.g. depression, Alzheimer's), inter alia, for deriving early indicators. Alternatively, the system is suitable for use in cognitive psychological test series. The system is designed, e.g., to examine and/or to determine an attention, a vigilance, a hearing effort, a cognitive stress, a cognitive processing ability and/or a sensory functionality of the user. Due to the special design of the system, these cognitive psychological test series can be performed in a domestic environment, in care facilities, but also at doctor's offices or in hospitals and clinics. A particular advantage of the system is, again, the simple installation of the electrodes due to the time saved. The system according to the invention is further suitable for use for detecting cognitive conditions as described above in work situations, in particular for safety-critical applications (e.g. train conductors, pilots, air traffic controllers, truck drivers, mining vehicles). A further possible application of the system is in the neuropsychological research and in adjacent research fields regarding cognitive conditions, but also in research fields regarding neurological disorder symptoms in a waking but also in a sleeping state.

According to an embodiment, the system comprises a hearing aid. The evaluation arrangement is configured to control the hearing aid based on the electric signals.

According to an embodiment, the evaluation arrangement is configured to correlate the signals measured by the device with an envelope of an audio signal recorded by the hearing aid, in order to control a beam former of the hearing aid. The inventors have recognized that the electric signals recorded by the device may indicate which audio signal the user is paying attention to. The system can therefore be configured to direct the beam former in the direction of the attention of the user in order to highlight the respective audio signal. The evaluation arrangement is configured, e.g., to recognize an attention of the user to a time signal, i.e. the audio signal, based on the correlation. The device is configured, e.g., to measure and/or to detect electric signals that are based on muscle movements of the eye and/or on the eye body which may function as a dipole. The evaluation device is configured, e.g., to generate an EOG based on the electric signals and to thus derive information on the viewing direction of the user and or on the blinking rate of the user. The viewing direction also indicates elements of attention, so that the system can thus be configured to direct the beam former into the direction of the attention of the user. Thereby, the hearing aid is configured to amplify, in a targeted manner, the audio signals from the direction of the attention of the user for the user. Thus, a system is provided that improves the hearing quality received by the user.

According to an embodiment, the evaluation arrangement is configured to evaluate the signals measured by the device for predetermined signal properties in order to control the hearing aid in response to signals of the user, such as, e.g., turning the hearing aid on and off, or also regulating the volume or particular parameter sets or also the signal amplification from a specific direction. Optionally, the evaluation arrangement comprises a database in which the predetermined signal properties are saved. Control commands for the hearing aid may be linked to the predetermined signal properties. The device may be configured, for example, to measure and/or detect electric signals based on muscle movements of the eye and/or on the eye body, which may function as a dipole. The evaluation arrangement may be configured to detect signal properties based on these signals, which signal properties are assigned, e.g., to a predetermined eye movement, such as, e.g., a wink or also an eyebrow movement. This is based on the knowledge that the signals measured by means of the device may indicated characteristic facial movements on the face surface and/or on the head of the user. Thus, the system makes it possible to control the hearing aid automatically with facial movements without having to actuate buttons on the hearing aid. This facilitates the handling of the hearing aid.

A further embodiment relates to a method for producing the device according to one of the embodiments described above. The method includes, inter alia, the step of providing the carrier structure, wherein the carrier structure is adapted to a face surface of a head of a user of the device, and wherein the carrier structure is shape-stable within a tolerance range without external force exerted by a user and is deformable under external force exerted by the user, in order to change a distance between two adjacently arranged electrodes and/or to area-comprehensively adapt to face and head structures. The shape-stability may be achieved, e.g. by using a material such as polyurethane foam material, for example, and a certain thickness, e.g., in a range of 0.5 mm to 2 mm, for the carrier structure. Alternatively, the carrier structure may be assembled of a carrier substrate and a removable stiffening element. The carrier substrate may comprise, e.g., a carrier material, wherein polyurethane material, polymer material, and/or silicone material is suitable. The removable stiffening element is configured, for example, to stabilize the shape of the carrier structure. According to an embodiment, at least one removable stiffening element may be arranged locally on the carrier substrate. Alternatively, the entire carrier substrate may also be covered by the stiffening element. A further step of the method relates to affixing an electrode arrangement on the carrier structure. The electrode arrangement comprises a plurality of electrodes for measuring electric signals on a face surface of a head of a user of the device and/or for measuring electric signals behind an ear of the user of the device. The carrier structure at least partially defines a relative position of the electrodes to one another, wherein the carrier structure is configured to position at least a part of the electrode arrangement on the face surface and/or behind the ear of the user.

A further embodiment relates to a method for using the device according to one of the embodiments described above. The method includes, inter alia, the steps of applying the device on a face surface of a head of a user by the user themselves or by another person, and monitoring the user based on the electric signals measured by the plurality of electrodes. A particular advantage of the present invention is that it may be applied by the user themselves.

According to an embodiment, the method comprises a monitoring of a sleeping behavior of the user based on the measured electric signals; or a monitoring of an epileptic behavior of the user based on the measured electric signals; or a monitoring of a neurological disorder of the user based on the measured electric signals; or a monitoring of a cognitive psychological behavior based on the measured electric signals.

According to an embodiment, the application of the device on the face surface of the head of the user comprises, inter alia, the steps of feeling an upper cheek bone and applying a first electrode on the upper cheek bone; applying a second electrode in front of the tragus of an ear of the user; applying a third electrode on a forehead, vertically above a nasion in an area of 1-3 cm above an eyebrow line; and applying a fourth electrode behind the ear. It is particularly advantageous if, in a first step, the first electrode and, in a second step, the second electrode are affixed on the face surface. This is based on the knowledge that the cheekbone and the point in front of the tragus of the ear are very distinctive points on a face surface and are therefore easy to find for a user. Thereby, the first and second electrodes can be applied on the face surface in a very precise manner. For applying the remaining electrodes of the plurality of electrodes of the device, the user is guided by a shape of the carrier structure of the device as the shape of the carrier structure is configured, e.g., to define the positions of the remaining electrodes relative to the first electrode and the second electrode on the face surface.

### BRIEF DESCRIPTION OF THE DRAWINGS

Embodiments of the present invention are subsequently described in more detail with reference to the accompanying drawings. With respect to the illustrated schematic drawings, it is to be noted that the illustrated functional blocks are to be understood both as elements or features of the apparatus according to the invention as well as corresponding method steps of the method according to the invention, and corresponding method steps of the method according to the invention may also be derived therefrom, in which:

FIG. 1a shows a schematic representation of device according to an embodiment of the present invention;

FIG. 1b shows a schematic representation of the device on a head of a user according to an embodiment of the present invention;

FIG. 2 shows an n=1 rotation;

FIG. 3a shows a schematic representation of a device with a stiffening element according to an embodiment of the present invention;

FIG. 3b shows a shape-stability of a connecting rib of a carrier structure with or without a stiffening element of a device according to an embodiment of the present invention;

FIG. 4a shows a schematic representation of a device with a connecting rib to a chin area of a user of the device according to an embodiment of the present invention;

FIG. 4b shows a schematic representation of a device with a carrier structure comprising bulges at certain electrode positions according to an embodiment of the present invention;

FIG. 5a shows a schematic representation of a device on a head of a user in a side view according to an embodiment of the present invention;

FIG. 5b shows a schematic representation of a device on a head of a user in a view from a back of the head of a user according to an embodiment of the present invention;

FIG. 6a shows a schematic representation of linear combinations of electrode channels for an EEG according to an embodiment of the present invention;

FIG. 6b shows a schematic representation of linear combinations of electrode channels for an EOG and EMG according to an embodiment of the present invention;

FIG. 7 shows a schematic representation of a device with cable connections according to an embodiment of the present invention;

FIG. 8a shows a schematic representation of an electrode integrated into a carrier structure of the device according to an embodiment of the present invention;

FIG. 8b shows a schematic representation of an electrode with a gel lens, integrated into a carrier structure of the device according to an embodiment of the present invention;

FIG. 9a shows a schematic representation of a device with a signal amplifier in a headband according to an embodiment of the present invention;

FIG. 9b shows a schematic representation of a signal amplifier in a collar according to an embodiment of the present invention;

FIG. 9c shows a schematic representation of a device with a signal amplifier behind or below an ear of a user according to an embodiment of the present invention;

FIG. 9d shows a schematic representation of a device with a signal amplifier on a shoulder or on a chest of a user according to an embodiment of the present invention;

FIG. 9e shows a schematic representation of a position of a signal amplifier relative to a muscle group in a neck region of a user according to an embodiment of the present invention;

FIG. 10 shows a block diagram of a method for using the device according to the invention;

FIG. 11 shows a block diagram of a system with an amplifier and a device according to the invention;

FIG. 12 shows a schematic representation of a system with an evaluation arrangement and a device according to the invention;

FIG. 13 shows a block diagram of a method for producing a device according to the invention;

FIG. 14 shows a conventional electrode placement in a polysomnography;

FIG. 15 shows a schematic representation of a device with an indication signal output on the carrier structure according to an embodiment of the present invention; and

FIG. 16 shows a schematic representation of a device with an electrode arrangement that is arranged behind an ear of a user according to an embodiment of the present invention.

### DETAILED DESCRIPTION OF THE INVENTION

Before embodiments of the present invention are subsequently described in more detail on the basis of the drawings, it is to be noted that identical or functionally identical elements, objects and/or structures or elements, objects and/or structures having the same effect are provided in the different figures with the same or similar reference numerals so that the description of these elements illustrated in different embodiments is interchangeable or maybe applied to one another.

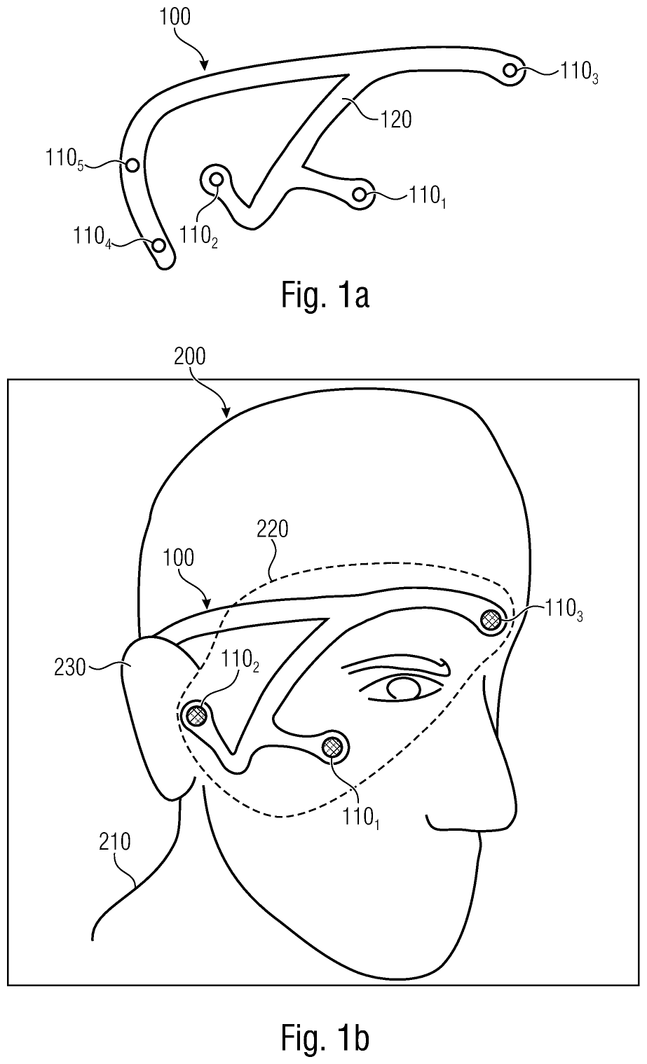

FIG. 1a shows a schematic representation of a device 100 according to an embodiment of the present invention and FIG. 1b shows the device 100 on a head 200 of a user 210.

The device 100 comprises an electrode arrangement 110 having a plurality of electrodes 110₁ to 110₅ for measuring electric signals on a face surface 220 of the head 200 of the user 210 and behind an ear 230 of the user 210. According to FIG. 1a and FIG. 1b, a first electrode 110₁, a second electrode 110₂, and a third electrode 110₃ are configured to measure electric signals on a face surface 220 of the head 200 of the user 210. A fourth electrode 110₄ and a fifth electrode 110₅ are configured to measure electric signals behind the ear 230 of the user 210. However, the device is not limited to the number and arrangement of the electrodes 110₁ to 110₅ represented in FIGS. 1a and 1b.

Furthermore, the device 100 comprises a carrier structure 120. The plurality of electrodes 110₁ to 110₅ are arranged, e.g., on a corresponding plurality of electrode positions on the carrier structure 120. The plurality of electrodes 110₁ to 110₅ of the electrode arrangement 110 are connected, e.g., via the carrier structure 120, wherein the carrier structure 120 at least partially defines a relative position of the electrode 110₁ to 110₅ to one another. The carrier structure 120 comprises, e.g., branching connecting ribs that connect the electrodes 110₁ to 110₅ to one another and at least partially define the relative positions of the electrodes 110₁ to 110₅ to one another as the positions of the plurality of electrodes 110₁ to 110₅ are limited to positions on the connecting ribs. The shape and length of a connecting rib between two adjacent electrodes defines, e.g., positions of the two electrodes on the head 200 of the user. In that, the shape follow, e.g., a human face shape and/or human facial structures. The shape of the carrier structure defines, e.g., a position on the head 200 of the user, i.e. on the face surface 220 and behind the ear 230, for each electrode of the plurality of electrodes.

The carrier structure 120 is configured to position at least a first part of the electrode arrangement, e.g., the electrodes 110₁ to 110₃, on the face surface 220 and to position a second part of the electrode arrangement, e.g., the electrodes 110₄ to 110₅, behind the ear 230. Due to its special shape, for example, the carrier structure 120 is configured for positioning the plurality of electrodes 110₁ to 110₅ as, e.g., the shape and length of the branching connecting ribs follows a face and/or head structure and thus, the branching connecting ribs define positions of the electrodes 110₁ to 110₅ on the face surface 220 of the head 200 of the user 210 and behind the ear 230 of the user 210.

The carrier structure 120 is shape-stable within a tolerance range without external force exerted by a user 210 and is deformable under external force exerted by the user 210. Shape-stable within a tolerance range means that, e.g., the carrier structure can only slightly bend, e.g., under the influence of gravity. Thereby, it is avoided that the shape collapses upon application on the head 200 by the user. The carrier structure is configured, e.g., to only allow bending of a maximum of 6 cm across a length portion of the carrier structure 120 of 10 cm without the user 210 applying external force. For this purpose, the carrier structure may comprise, e.g., a support structure that is configured to counteract a deformation of the carrier structure. This support structure may be designed as a removable stiffening element so that it can be removed after affixing the carrier structure on the head surface. Alternatively, the shape-stability may also be achieved by means of the thickness of the carrier structure. The thicker the carrier structure the more shape-stable it is. With a thickness of 1 mm, for example, a sufficient shape-stability is achieved. With lower thicknesses, the shape-stability may be achieved with the aid of the support structure. Additionally to the shape-stability, the carrier structure 120 exhibits a certain stretchability or elasticity, whereby the carrier structure can be deformed under external force exerted by the user 210. The user may, for example, stretch or bend the carrier structure to change a distance between two adjacently arranged electrodes and/or to area-comprehensively adapt to face and head structures. Due to the area-comprehensive adaptation, the carrier structure comprises a larger contact surface on the head surface, whereby the carrier structure can be affixed in a more robust manner on the head surface of the user. The carrier structure is thus configured to stably remain on the skin over several hours or over an entire night or over an entire day or multiple days. The carrier structure is configured, e.g. to increase the distance between two adjacently arranged electrodes in a range of 5% to 10%, or 5% to 20% under the external force exerted by the user 210. Usually, this is particularly exploited only for reaching the forehead electrode, e.g., the third electrode 110₂. The flexibility of the material, however, is advantageous for being able to perform minor adjustments of the course over and behind the ear 230 and to facilitate a high wear comfort in the movement in the face.

For the selection of the material of the total structure, it is important, e.g., that
(a) the carrier structure 120 adhering to the skin is easily deformable (non-rigid and/or stretchable in parts) under the influence of the user in order to adapt well to facial structures (e.g., bulges) for a planar and thus robust adhesion and to proportional differences of the face for achieving the desired electrode positions; and that
(b) the carrier structure 120 remains shape-stable for the adhesion process without the influence of the user so that when sticking on the device, the arms of the grid (e.g. the net of branching connecting ribs of the carrier structure) do not inadvertently stick to parts of the face, fingers, or to one another when they hang down due to a very high non-rigidity. po The carrier structure 120, which ultimately sticks to the skin, is therefore characterized by stretchability and/or non-rigidity, i.e. shape-instability or shape-lability:

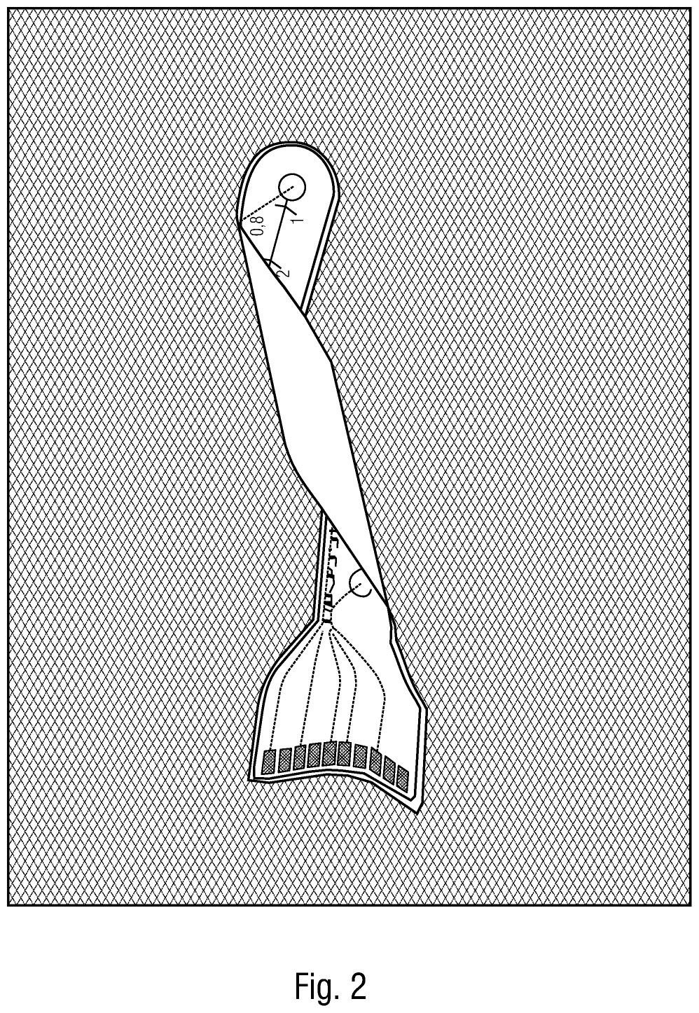

The carrier structure is, e.g., deformable such that a connecting rib of the carrier structure 120 can be twisted in itself multiple times without the connecting rib tearing. In FIG. 2, such a twisting is shown with paper with n=1 twists or rotations. Such a deformability may be achieved, for example, if the carrier structure comprises polyurethane material, polymer material, and/or silicone material as the carrier material. Furthermore, electrical connections of the plurality of electrodes to a signal output of the device may have a further influence on the deformability of the carrier structure. In order to retain the stretchability of the total structure, traces and/or cable connections are designed, e.g., such that a length supply, e.g., additional lengths, is/are available in the form of waves or loops. If the carrier structure comprises, e.g., a polyurethane material as the carrier material, this may be present, e.g., in the form of polyurethane film material in which traces with a slung course are inserted, or be present, e.g., in the form of poly urethane foam on which cable connections are arranged. In the case of the polyurethane film material, the carrier structure is configured, e.g., to enable and/or allow for a stretching in a range of 5% to 10%, and in the case of polyurethane foam, the carrier structure is configured, e.g., to enable and/or allow for a stretching in a range of 5% to 20%.

A thin (e.g. with a thickness of between 0.1 mm and 0.5 mm) polyurethane film material may be twisted in itself multiple times as a material strip without the material tearing. The carrier material proposed here as a thin film is supposed to withstand more than n=1 twists without tearing or failing otherwise. The non-rigidity leads to it being very easily (slight pressure of the fingers, i.e. under external force exerted by the user 210) deformable in any desired direction and thus leads to a high wear comfort as the edges of the carrier structures thus easily fit snug on the head surface. Moreover, it comprises a stretchability of up to 5% or up to 10%, depending on, e.g., the selection of a trace geometry or depending on the cable routing along the carrier structure. The high non-rigidity may call for a removable stiffening element on the side of the carrier structure facing away from the skin on order to obtain the stiffness needed for the application process, wherein the stiffening element is, e.g., part of the carrier structure. After or during the application, this additional stiffening element may be removed in order to make use of the low thickness and the full stretchability for the wear comfort.

Polyurethane foam with a thickness of up to 1 mm does not comprise a non-rigidity as high as that of the thin polyurethane film material, but is, in turn, very stretchable (up to 20% of the length) and can also be deformed very easily (slight pressure of the fingers) and thus also meets the requirements posed herein.

In general, a material within the meaning of this application is non-rigid if it is deformable and/or bendable in all directions and can be twisted without being damaged, e.g., without tearing. Non-rigidity may be understood to mean, e.g., that a film and/or layer of the material already bends with only the influence of gravity, i.e. without further external force application or with only slight external force application (e.g., F≤N). The non-rigidity allows for a planar adaptation to any unevenness of face and head structures. If the structure (e.g. the carrier structure with a polyurethane film having a maximum thickness of 0.1 mm, 0.3 mm, or 0.5 mm or the carrier structure with polyurethane foam having a thickness in a range of 0.5 mm to 2 mm) is placed on a radial elevation (assumed herein as a cylinder) with a diameter of, e.g., 0.5 cm and a height of 1 cm, the carrier material may simultaneously come to lie, either following gravity due to its own weight or due to minimal force application (<1 N), in each case, on the circle surface and on four locations of the lateral surface of the cylinder, of which, in each case, two of the areas on the lateral surface are opposite one another and the connecting lines of the respectively opposite surfaces are perpendicular to one another, without the carrier structure being damaged (tearing or breakage). Here, the structure follows the 90° corner of the area of the circle surface to the lateral surface of the cylinder with a tightly fitting radius of a maximum of 1 mm. If a layer or structure is non-rigid, it follows, for example, any unevenness. Non-rigid structures and layers are characterized, e.g., by a low elastic modulus, low tensile stiffness, and thus great deformations following even low force and momentum loads. Non-rigidity may be understood to mean, e.g., that a structure is deformable in a thickness direction of the structure simultaneously along two lateral directions perpendicular to one another without being damaged and/or that the structure can be twisted multiple times without being damaged, i.e. when a torsional moment, for example, acts on the structure in order to twist the same multiple times about an axis perpendicular to the thickness direction.

The total structure is intended to remain shape-stable without the influence of the user 210 so that the device 100 can be easily applied by the user 210 themselves. If the carrier structure comprises, for example, the thin polyurethane film material, the carrier structure optionally further comprises further materials or elements that are configured to stabilize the carrier structure and thus ensure the shape-stability of the carrier structure. These reinforcing materials or reinforcing elements are, e.g., arranged in a removable manner on the thin polyurethane film material so that the carrier structure is shape-stable if it comprises a polyurethane film material and a reinforcing material or a reinforcing element, and that the carrier structure is non-rigid if the reinforcing material or the reinforcing element is removed from the polyurethane film material. However, in the case of polyurethane foam, no support structure is needed as the material itself already exhibits a shape-stability while being stretchable upon influence by the user. Optionally, however, the carrier structure may also be further stabilized in the case of a carrier material with polyurethane foam.

The shape-stability of the carrier structure in the case of no external force exerted by the user 210 may be achieved in different ways:

According to an embodiment, the carrier structure 120 comprises an adhesive material for affixing the plurality of electrodes 110₁ to 110₅ and the carrier structure 120 on the face surface and behind the ear 230. The adhesive material is, e.g. covered by a removable protective layer. The protective layer comprises, e.g., a film or paper material. The protective layer comprises, e.g., a plurality of protective portions that can be independently removed from the carrier structure 120. The film or paper material for covering the adhesive layer and/or the adhesive material may contribute additional stability as, during the application of the carrier structure 120 on the head 200, the film or paper material can be peeled off in individual passages, i.e. in individual protective layer portions, and thus, stability can be sustained during the process.

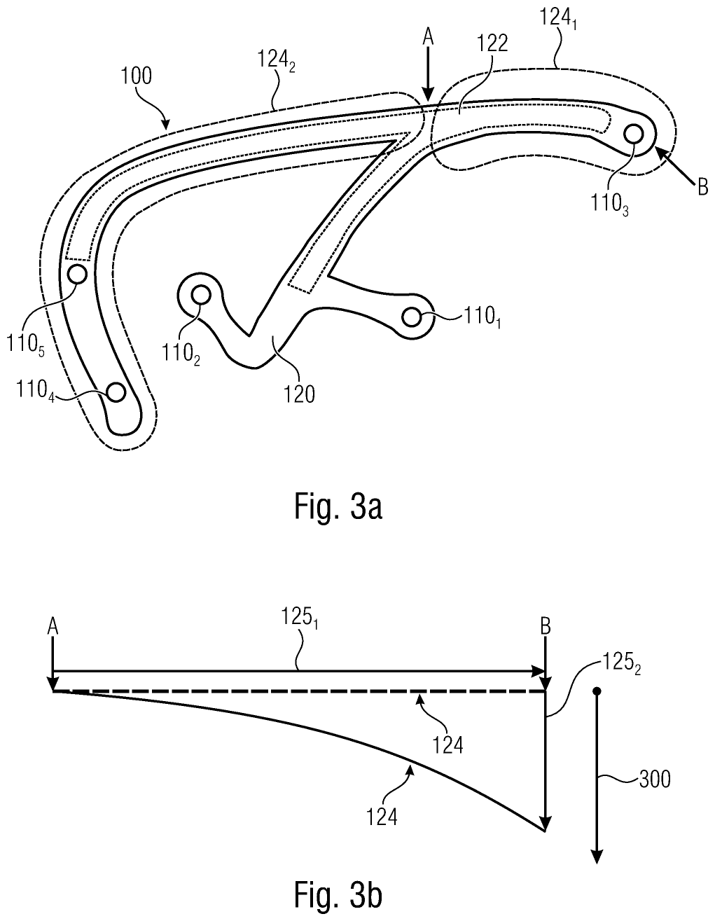

In addition or as an alternative thereto, it is also possible for a support structure, e.g., in the form of a film (e.g. made of a polyimide material, polyester material, polyethylene material or FR4 material) or paper or polyurethane foam, to be applied to the side of the carrier structure 120 facing away from the skin. The support structure may also be considered a stiffening element 122. As shown in FIG. 3a, the carrier structure 120 may comprise a removable stiffening element 122 on a side facing away from the electrode arrangement 110, which stiffening element 122 is configured to at least locally stiffen the carrier structure 120. The stiffening element 122 is present, e.g., only during an application process of the carrier structure 120 on the head surface, i.e. on the face surface 220 and behind the ear 230. After or while sticking on and/or applying the carrier structure 120, which may occur, as explained above, also in parts, the stiffening element 122 may be peeled off the carrier structure 120. This is relevant particularly for thin carrier materials with a very high non-rigidity as the stiffening element 122 counteracts the non-rigidity of the carrier material and thus substantially facilitates an autonomous application of the device 100 on a head surface of the user 210. By the stiffening element 122 being removable from the carrier structure after the application of the carrier structure 120 on a head surface of the user 210, the wear comfort for the user 210 is increased as the carrier material is deformable without the stiffening element 122 and is thus configured to deform along with movements in the face, such as, e.g., muscle movements or also jaw movements. The adhesive bond between the stiffening element 122 and the carrier structure 120 is configured, e.g., to not leave behind adhesive remnants when removing the stiffening element.

In FIG. 3a, a device 100 is shown which may comprise all features and functionalities that have also been described in the context of the device of FIGS. 1a and 1b. FIG. 3a shows a stiffening element 122 arranged on a branching connecting rib of the carrier structure 120. The connecting rib is configured, e.g., to connect the first electrode 110₁ and the second electrode 110₂ to the third electrode 110₃, on the one side, and to the fourth electrode 110₄ and the fifth electrode 110₄, on the other side. The connecting rib branches off, wherein a first part 124₁ of the connecting rib leads to the third electrode 110₃, and a second part 124₂ of the connecting rib leads to the fourth electrode 110₄ and the fifth electrode 110₅. Due to the stiffening element on the connecting rib, the shape of the connection between the electrodes is maintained. This is important particularly for positioning the carrier structure 120 on the head surface as the shape of the connecting rib at least partially defines the relative position of the electrodes to one another and is configured to position the electrodes on the head surface such that the electrodes assume predetermined positions on the head surface. As an alternative to the one stiffening element 122, the device 100 may also comprise multiple stiffening elements or also one stiffening element 122 that covers the entire surface of the carrier structure 120 (i.e. the surface on the side facing away from the electrode arrangement 110).

A further possibility is to embody the carrier structure with a greater thickness. If the carrier structure 120 comprises, e.g., polyurethane foam material as the carrier material, this may be embodied, e.g., having a thickness in a range of 0.5 mm to 2 mm in order to achieve the shape-stability of the carrier structure without an additional stiffening element 122. However, in this case, as well, the carrier structure 120 may optionally comprise an additional stiffening element.

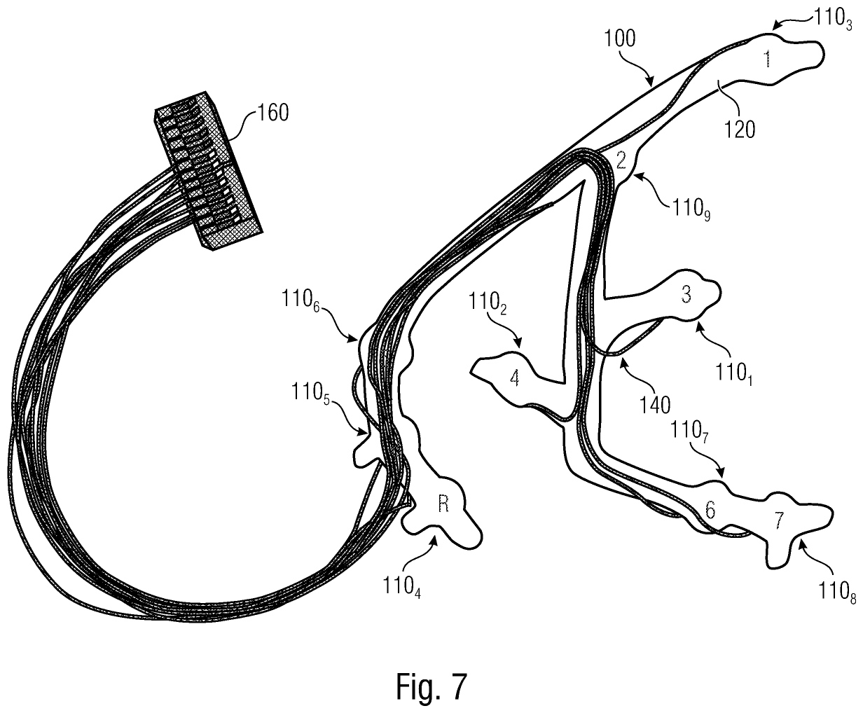

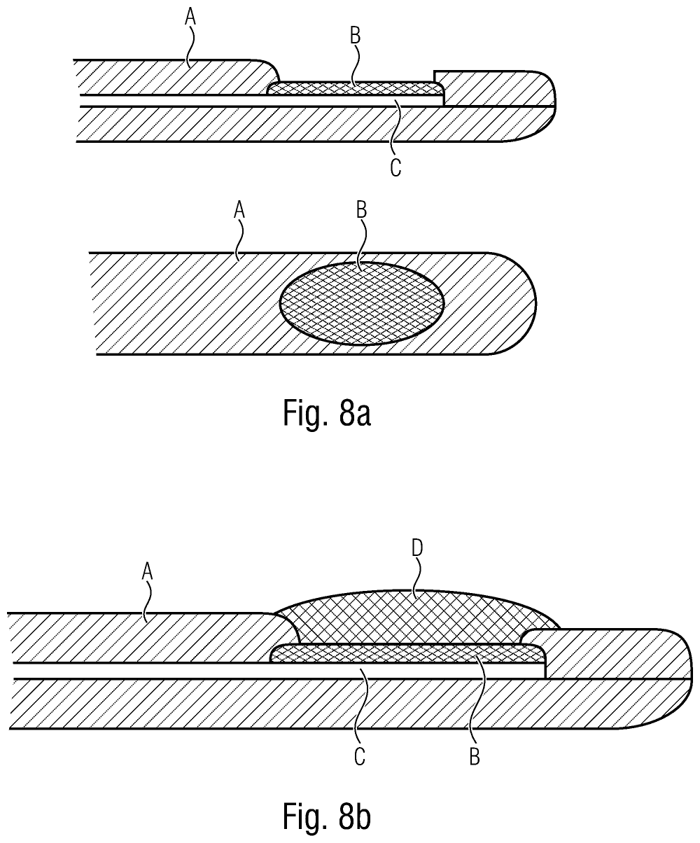

Polyurethanes, as a thin carrier film with a then correspondingly applied additional, temporary support structure, i.e. with the stiffening element 122, or as polyurethane foam for thicknesses in a range of 0.5 mm to 2 mm, are particularly suitable as materials for the carrier structure 120. Advantageously, the traces are directly applied to the polyurethane film and/or the traces (excluding the electrode surfaces) are inserted into a polyurethane sandwich construction (cf. FIGS. 8a and 8b with the accompanying description), the polyurethane foam design may also serve as a carrier structure for conventional solid gel electrodes with cables (cf. FIG. 7 with the accompanying description).

The shape-stability of the grid structure, i.e. the carrier structure 120, with a corresponding support structure, i.e. with the stiffening element 122, is shown by way of example in FIG. 3b in how an arm 124, i.e. a connecting rib, of 10 cm of length (see 125₁) tilts downwards, following gravity 300, when the arm 124 is oriented to be horizontal (perpendicular to gravity 300). In this length, the arm 124 does not tilt any further than about 6 cm (see 125₂). If this is applied, e.g., to the connecting rib 124₁ of FIG. 3a, the connecting rib 124₁ comprising a length of about 7 cm between points A and B (see 125₁) does not tilt further than about 4 cm (see 125₂). In general, a connecting rib of the carrier structure 125 is configured to be shape-stable within a tolerance range without external force exerted by the user 210. A horizontally oriented connecting rib of the carrier structure 120 is configured, e.g., to tilt, at a length of up to a maximum of 12 cm under the influence of gravity 300, a maximum of 60% of its length, following gravity. Limiting the tilt and/or bending may be achieved, inter alia, by means of the features discussed above, such as an adhesive protective film, stiffening element 122, or a special thickness of the carrier structure. During the application, the grid, i.e. the carrier structure, is kept upright so that the arm 124 tilts even less as it is situated at a different angle relative to gravity 300. FIG. 3b therefore shows by way of example a stiffness of the carrier structure 120 with a support structure and/or an effect of the support structure on the shape-stability of the carrier structure 120.

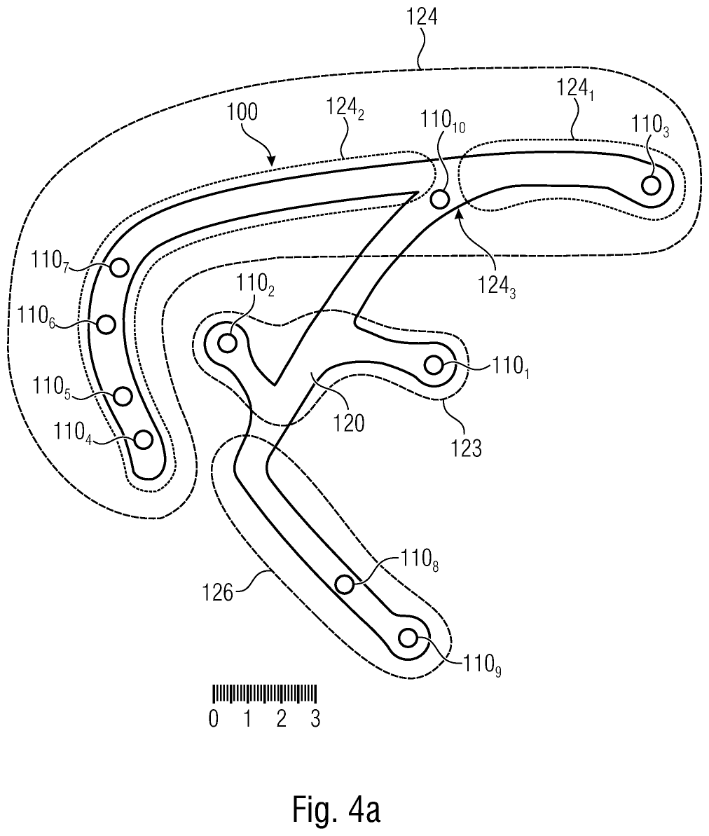

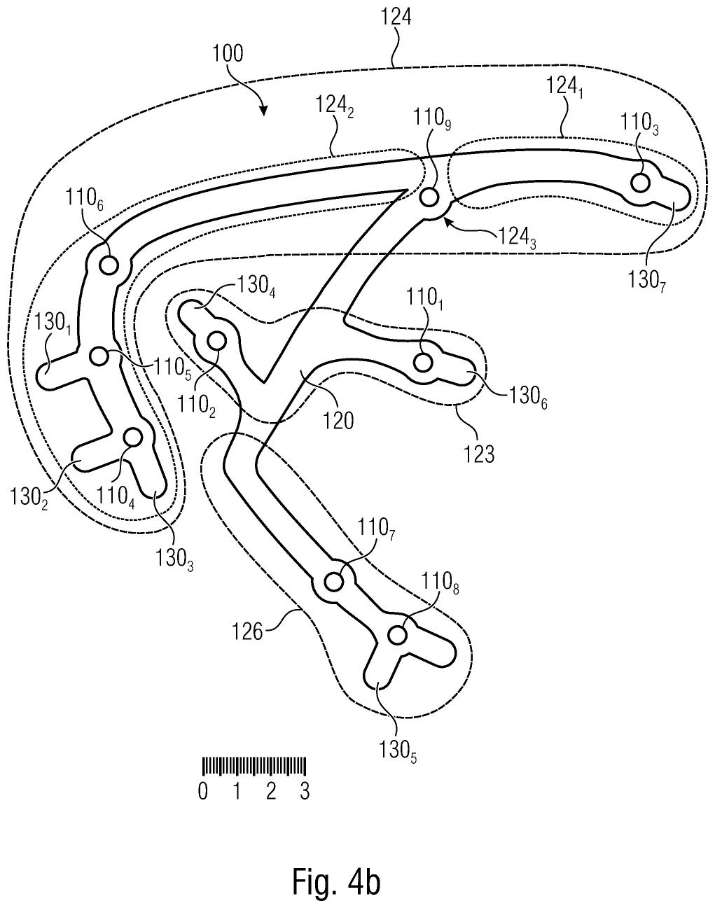

FIGS. 4a and 4b show a further embodiment of the device 100 according to the invention. Optionally, the device may comprise features and functionality as these have been described in connection with embodiments of FIGS. 1a to 3b. Likewise, it is possible that embodiments of FIGS. 1b to 3b comprise features or functionalities, as they will be described below. The device in FIG. 4a is different from the device in FIG. 1a merely in that more electrodes are arranged on the carrier structure 120, and the carrier structure 120 further comprises a connecting rib connecting the first electrode 110₁ and the second electrode 110₂ to electrodes whose positions may be assigned to a chin of the user. The device in FIG. 4b is different from the device in FIG. 4a merely in the number and the diameter of the electrodes and in bulges of the carrier structures at certain electrode positions.

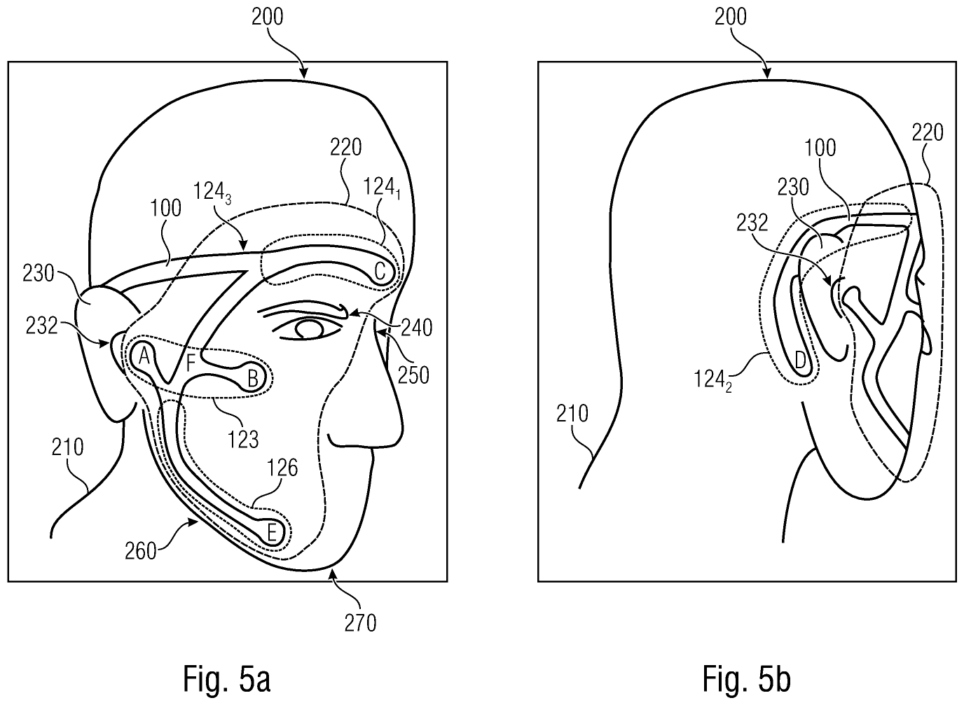

As shown in FIG. 4a, the shape of the carrier structure 120, on which the plurality of electrodes 1101 to 11010 (and/or 1101 to 1109 in FIG. 4b) is arranged, constitutes a particular feature of the present invention. The shape is guided by the structures of a head 200 of the user 210. In FIGS. 5a and 5b, the device 100 of FIG. 4a and FIG. 4b is arranged, by way of example, on the head 200 of the user 210. FIG. 5a shows a placement of the device 100 in a side view of the head 200 of the user 210, and FIG. 5b shows a placement of the device 100 in a view from a back of the head of the user 210.

The carrier structure comprises, e.g., a first connecting rib 123 that connects the first electrode 110₁ and the second electrode 110₂, wherein the first electrode 110₁ is arranged at a first end of the first connecting rib 123, and the second electrode 110₂ is arranged at a second end of the first connecting rib 123. The first connecting rib 123 comprises, e.g., a wound and/or slung course, such as a wavelike course.

Due to the slung course of the connecting rib between the first electrode 110₁ and the second electrode 110₂, a very shape-stable connection between the two electrodes is realized. This is advantageous particularly because the first electrode and/or the second electrode may constitute a reference for the positions of the remaining electrodes of the plurality of electrodes 110₁ to 110₁₀ (or 110₁ to 110₉ in FIG. 4b) and thus, it is ensured that the user can autonomously affix these two electrodes very precisely on the face surface 220. Furthermore, the special course of the first connecting rib 123 increases the wear comfort. This is due to a position of the first electrode 110₁ on the face surface 220 corresponds to, e.g., a position on a cheek bone, i.e. on a first distinctive point on the face surface, and a position of the second electrode 110₂ on the face surface 220 corresponds to, e.g., a position in front of a tragus 232 of the ear 230, i.e. on a second distinctive point of the face surface. Due to the slung course of the first connecting rib 123 and due to the deformability and/or elasticity of the carrier material of the carrier structure 120, it is configured to follow facial movements in an area between the first electrode 110₁ and the second electrode 110₂. This increases the wear comfort.

According to an embodiment, the carrier structure 120 comprises, e.g., a branching second connecting rib 124 that connects the first electrode 110₁ and the second electrode 110₂ to a third electrode 110₃ on one side, and to a fourth electrode 110₄ on the other side (the fourth electrode may also be one of electrodes 110₅ to 110₇ in FIG. 4a, or the electrode 110₅ or 110₆ in FIG. 4b), wherein the second connecting rib 124 branches off into a first part 124₁ and a second part 124₂ at a branching point 124₃.

The first part 124₁ of the second connecting rib 124 is configured to connect the first electrode 110₁ and the second electrode 110₂ to the third electrode 110₃. The second connecting rib 124 leads, e.g., from about a center of the first connecting rib 123 via the first part 124₁ to the third electrode 110₃, wherein the third electrode 110₃ is arranged at an end of the first part 124₁. The second connecting rib 124 runs from a center of the first connecting rib 123 via the first part 124₁ in an arc and/or in a curved shape. The second connecting rib follows, together with the first part 124₁, e.g., a shape of an eye and/or an eyebrow 240. If the carrier structure 120 is arranged on a head surface of the user, the second connecting rib 124 runs above, along the eyebrow 240, to the third electrode 110₃. The position of the third electrode 110₃ corresponds, on the face surface 220, e.g., a position vertically above a nasion 250 of the user 210, i.e. a third distinctive point on the face surface 220. In other words, the carrier structure comprises a connecting rib, namely the second connecting rib 124 that at least partially connects a first electrode position that is assigned to the first electrode 110₁ of the plurality of electrodes, and/or a second electrode position that is assigned to the second electrode 110₂ of the plurality of electrodes, on one side, to a third electrode position that is assigned to the third electrode 110₃ of the plurality of electrodes, on the other side, and wherein the third electrode position matches, within a tolerance range, defined by the carrier structure 120, of 1 to 2 cm to on both sides of a position on a forehead of the face surface, in an area of 1 cm to 3 cm above the eyebrow line vertically above a nasion 250 of the user.

The second part 124₂ of the second connecting rib 124 is configured to connect the first electrode 110₁ and the second electrode 110₂ to the fourth electrode 110₄. Optionally, further electrodes (e.g., electrodes 110₅ to 110₇ in FIG. 4a, or electrodes 110₅ to 110₆ in FIG. 4b) of the plurality of electrodes may be arranged on the second part 124₂ of the second connecting rib 124, which is why the second part 124₂ of the second connecting rib 124 may further be configured to connect the first electrode 110₁ and the second electrode 110₂ to the further electrodes. The second part 124₂ of the second connecting rib 124 runs, e.g., from the branching point 124₃ in an opposite direction from the first part 124₁ of the second connecting rib 124. At an end of the second part 124₂, the second connecting rib 124 is, e.g., curved and/or extends in an arc. The arc of the second connecting rib runs, e.g., around the position of the second electrode 110₂. If the carrier structure 120 is arranged on the head surface of the user 210, the arc of the second connecting rib 124 runs, e.g., along the shape of the ear 230. The fourth electrode 110₄ as well as possible further electrodes of the plurality of electrodes are advantageously arranged on the arc of the second connecting rib 124. The position of the fourth electrode 110₄ corresponds, on the head surface, e.g., to a position behind the ear 230 of the user 210. Optionally, the position of the fourth electrode corresponds to the position of the mastoid (cf. point D in FIG. 5b), i.e. a fourth distinctive point on the head surface.

In other words, the carrier structure comprises a connecting rib, namely the second connecting rib 124, that at least partially connects the first electrode position and/or the second electrode position, on one side, to a fourth electrode position that is assigned to the fourth electrode 110₄ of the plurality of electrodes, on the other side, and wherein the fourth electrode position corresponds, within a tolerance range determined by the carrier structure 120, to a position behind the ear 230 on a mastoid of the user 210. The second part 124₂ of the second connecting rib 124 leads, e.g., above the ear 230 of the user 210, to an arc that follows the shape of the ear 230 behind the ear 230.

According to an embodiment, an electrode (e.g. 110₁₀ in FIG. 4a, or 110₉ in FIG. 4b) of the plurality of electrodes may be arranged in an area of the branching point 124₃ of the second connecting rib 124. Thereby, the first electrode 110₁, the third electrode 110₃, and the electrode 110₁₀/110₉ form, e.g., a triangle in the area of the branching point 124₃ of the second connecting rib 124. The triangle comprises a right angle, with a tolerance range of ±5°, at the electrode 110₁₀/110₉ in the area of the branching point 124₃ of the second connecting rib 124. Due to the special arrangement of these three electrodes, an eye movement can be detected very precisely using the device 100.

Optionally, the carrier structure 120 further comprises a third connecting rib 126 that connects the first electrode 110₁ and the second electrode 110₂ to a fifth electrode (e.g. 110₉ in FIG. 4a, or 110₈ in FIG. 4b). The third connecting rib 125 runs, e.g., from about the center of the first connecting rib 123 in an opposite direction from the second connecting rib 124, wherein the fifth electrode 110₉/110₈ is arranged at an end of the third connecting rib 126. The third connecting rib 126 comprises, e.g., a curved course. If the carrier structure 120 is arranged on a face surface 220 of the user 210, the third connecting rib 126 runs along a jaw 260 to the fifth electrode 110₉/110₈. The position of the fifth electrode 110₉/110₈ corresponds, on the face surface 220, e.g., to a position on or by a chin 270 of the user 210, i.e. a fifth distinctive point on the face surface 220. Optionally, further electrodes of the plurality of electrodes may be arranged on the third connecting rib 126. In other words, the carrier structure 120 comprises a connecting rib, namely the third connecting rib 126, that at least partially connects the first electrode position and/or the second electrode position, on one side, to a fifth electrode position that is assigned to the fifth electrode 110₉/110₈ of the plurality of electrodes, and wherein the fifth electrode positions corresponds, within a tolerance range determined by the carrier structure 120, to a position on the chin 270 of the face surface 220 of the user 210.

The connecting ribs of the carrier structure 120 comprise, e.g., a rib width that corresponds at most to three times, two times, or 1.5 times the electrode diameter of an electrode of the plurality of electrodes. Transition areas, i.e. the connecting ribs, may also be designed as a “slim waist” in order to achieve an optimal flexibility for adapting to different head shapes.

The first electrode 110₁ and/or the second electrode 110₂ define/defines, for example, a reference position, and the remaining electrodes of the plurality of electrodes are arranged on the carrier structure 120 relative to the reference position. According to an embodiment, a first electrode position corresponding to the position of the first electrode 110₁ on the carrier structure 120 may serve as a reference position, or a second electrode position corresponding to the position of the second electrode 110₂ on the carrier structure 120 may serve as a reference position. Alternatively, it is also possible that the remaining electrodes of the plurality of electrodes are arranged relative to the first electrode position and the second electrode position on the carrier structure 120. In this case, e.g., a position in the middle of the two electrode positions could serve as a reference position. As already mentioned above, the positions of the first and second electrodes serve particularly well as reference positions as they can be positioned very precisely by the user on their head surface. Based on these two positions, the carrier structure may define the positions of the remaining electrodes of the plurality of electrodes on the head surface, i.e. on the face surface 220 and behind the ear 230, very precisely, e.g., by means of the second connecting rib and by means of the optional third connecting rib.

According to an embodiment, the carrier structure 120 comprises an adhesive material for affixing the plurality of electrodes and the carrier structure 120 on the head surface, i.e. on the face surface 220 and behind the ear 230. The adhesive material is applied, e.g., to the side of the carrier structure 120 on which the electrode arrangement is arranged, as well. The adhesive material is thus arranged on the side of the carrier structure facing the skin of the user. Optionally, the electrodes of the plurality of electrodes may comprise an adhesive electrolyte gel in addition to or as an alternative to the adhesive material, which gel is configured to affix the plurality of electrodes on the head surface and to lower the impedance. The adhesive material and/or the adhesive electrolyte gel is covered by a protective film at least in some areas.

The protective film may be, for example, subdivided into a plurality of protective film portion that can be independently removed from the carrier structure 120 and/or the plurality of electrodes. A first protective film portion of the plurality of protective film portions covers, e.g., a first subset of the plurality of electrodes and of the carrier structure 120, and a second protective film portion of the plurality of protective film portions covers, e.g., a disjoint second subset of the plurality of electrodes and of the carrier structure 120. The first protective film portion covers, e.g., the first connecting rib 123 comprising the first electrode 110₁ and the second electrode 110₂. The second protective film portion covers, e.g., the second connecting rib 124 comprising, inter alia, the third electrode 110₃ and the fourth electrode 110₄ and optionally further electrodes of the plurality of electrodes. Optionally, the second connecting rib 124 may also be covered by two different protective film portions. A protective film portion (e.g., the second protective film portion) then covers, e.g., that part of the second connecting rib 124 which is in contact with the first connecting rib 123 and further comprises the first part 124₁ of the second connecting rib 124, and a further protective film portion (e.g., a third protective film portion) then covers, e.g., only the second part 124₂ of the second connecting rib. An optional further protective film portion covers, e.g., the third connecting rib 126 comprising at least the fifth electrode 110₉/110₈ of the plurality of electrodes. This division of the protective film portions is particularly advantageous as the protective film portion can always be removed from that area that is the next to be attached to the head surface of the user 210, and thereby, the device is easy to handle during application because the adhesive material is always exposed only for a small area. Furthermore, the special selection of the first protective film portion allows the user during the application to first focus completely on the positioning of the first electrode and the second electrode, whereby these can be positioned very precisely. The positions of the remaining electrodes of the plurality of electrodes on the head surface then result from the shape of the carrier structure 120, in particular from the shape of the second connecting rib 124 and the optional third connecting rib 126. Thus, the plurality of protective film portions leads to a high accuracy in positioning the device 100 and furthermore, allows for an autonomous positioning and affixing of the same on the head surface of the user 210 by the user 210.

Furthermore, extensions 130₁ to 130₇ and/or lugs as shown, e.g., in FIG. 4b are advantageous in positioning and affixing the device 100 on the head surface of the user 210. The carrier structure 120 comprises at least one extension 130₁ to 130₇, e.g., at a position at which an electrode of the electrode arrangement 110 is arranged. As is shown, e.g., for the fourth electrode 110₄, the carrier structure 120 may also comprise multiple extensions, for example two extensions, at a position at which an electrode of the electrode arrangement 110 is arranged. An adhesive material, for example, is arranged on the extensions, whereby affixing the electrodes, at the positions of which the carrier structure comprises at least one extension 130₁ to 130₇, on the face surface can be improved.

The positions of the electrodes of the electrode arrangement 110 were selected such that, on the one hand, only skin patches free of hair are used for affixing e.g. an adhesive carrier substrate, and, on the other hand, signal portions of the PSG electrodes to be used in a classic manner result from linear combinations of the electrode signals. The approach for forming the linear combinations is demonstrated in [Da Silva Souto, C. F., Pätzold, W., Wolf, I., Paul, M., Matthiesen, I., Bleichner, M. G., & Debener, S. (2021). “Flex-printed ear-EEG sensors for adequate sleep staging at home.”; Frontiers in Digital Health, 3, 66] based on the flexible electrode solution cEEGrid.

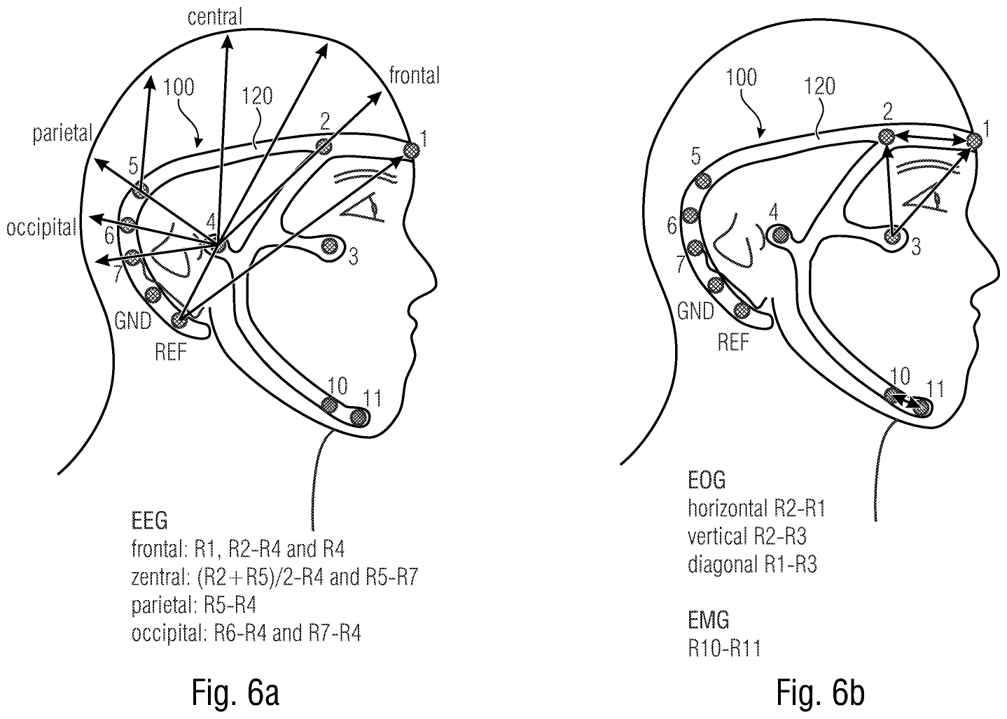

As shown in FIGS. 6a and 6b, the shape of the carrier structure 120 is configured, e.g., to position the plurality of electrodes on a corresponding plurality of positions on the face surface; wherein the plurality of positions is configured for detecting the electric signals on the face surface for at least one linear combination of the electric signals for mapping signals from a predetermined head region of the user. By means of the linear combination, it is possible, e.g., to derive an EEG, an EOG, and an EMG for a head region of the user. Signals of an EKG also show in the electrode channels and can be extracted via signal processing methods, e.g., the independence analysis (i.e. an Independent Component Analysis (ICA)). The linear combination of the electrode channels can be used, e.g., to highlight specific signal portions. The electrode channels are referred to as Rᵢ for i=1 to 11 and are referenced to the electrode REF.

FIG. 6a shows, by way of example, specific brain regions for which signals can be mapped by forming specific linear combinations. The electroencephalogram (EEG), e.g., is configured for a frontal region, for a central region, for a parietal region, and for an occipital region. For the frontal region, e.g., the signals of the electrode channels 1 and 4 are read directly, and the signals of the electrode channels 2 and 4 are linked to one another via a linear combination (R2−R4). For the central region, e.g., linear combinations of the signals of the electrode channels 1, 4, 5, and 7 (e.g. a first linear combination with (R2+R5)/2−R4 and a second linear combination with R5−R7) are formed. For the parietal region, e.g., a linear combinations of the signals of the electrode channels 4 and 5 may be formed (R5−R4), and for the occipital region, e.g., linear combinations of the signals of the electrode channels 4, 6, and 7 (e.g. a first linear combination with R6−R4 and a second linear combination with R7−R7) are formed. Thus, relevant sleep parameters can be mapped in the EEG despite a limited electrode placement.

FIG. 6b shows that, moreover, an electrooculogram EOG (vertical R2−R3), horizontal (R2−R1), and diagonal (R1−R3)) can be derived and an electromyogram EMG can be derived, particularly in an area of the chin (R10−R11). The electrodes of the electrode arrangement that are arranged around the eye of the user are arranged in a right triangle, e.g., within a tolerance range of ±5°. The eye movement is thus detected diagonally via the electrodes 1 and 3, horizontally via the electrodes 1 and 2, and vertically via the electrodes 3 and 2. The electrodes 10 and 11 serve for detecting an EMG.

Further applications for these electrode positions are conceivable, however, the arm towards the chin electrodes may also be omitted in the design. In a conceivable reduction to only the arm D (reference from FIG. 5a and FIG. 5b) behind the ear and C on the forehead, the pre-placement and possible rotation can take place via the electrode 2 (FIGS. 6a and 6b).

The signals also contain, e.g., information of an electrocardiogram. They can be extracted using signal processing methods, e.g., the Independent Component Analysis (ICA).

According to an embodiment, the user may wear a first carrier structure according to the invention with an electrode arrangement on one half of the face and a second carrier structure according to the invention with an electrode arrangement on an opposite half of the face. Thereby, the signals of the two electrode arrangements can be combined, allowing further linear combinations running centrally through the head.

In the devices 100 described herein, the plurality of electrodes may comprise wet electrodes and/or dry electrodes. It is of advantage for the plurality of electrodes to comprise only one type of electrode. In particular, wet electrodes are of advantage. Wet electrodes that are self-applicable by an untrained person widen the field of use of the wet electrodes for gathering electrophysiological signals, in particular EEG, but also EOG and EMG, significantly in a wide field of application. Thus, people are able to perform a high-quality gathering of data in a domestic environment without the presence of a trained person. Due to the wear comfort and discrete placement, a long-term measurement for monitoring biosignals even in day-to-day life is conceivable. With the device 100, a configuration of wet electrodes is brought into a grid structure, i.e. arranged on the carrier structure, such that they can be applied autonomously by an untrained person and reliably assume, with a low positional variance, certain electrode positions in the face and behind the ear.

The design of the electrode grid, i.e. the carrier structure 120 with the electrode arrangement 110, was based, inter alia, on the following requirements, for example:

It should be easy to apply autonomously, i.e. untrained people may apply the grid directly themselves without problems based on a manual or something similar. It is intended to fit different head shapes, i.e. the configuration is suitable for different head sizes and head shapes of adolescents and adults. The electrodes come to lie in a certain positional configuration on the face and behind the ear at specific positions. The wear comfort is intended to make a comfortable wearing over several hours to day possible. The signal quality is intended to be highly suitable for EEG, EMG, EKG, and EOG measurements with impedance values in the range of few kOhm to about 20 kOhm. More than 3 electrode positions are to be mapped in the grid formation, FIG. 4a shows, e.g., 9 (plus reference REF and ground GRD for the differential measurement of the potential differences), wherein more are possible at the positions of the arm embodiments or smaller side arms. The grid is available, e.g., in variants, in a similar shape embodiment, but made of different materials and/or embodiments of the electrodes.

A first variant of the device 100, as it is shown, e.g., in FIG. 7, comprises a solid gel grid with solid gel electrodes. Here, e.g., commercially available EKG electrodes 110₁ to 110₉ for the neonatal field may be inserted in a carrier structure. As an alternative to the solid gel electrodes, dry electrodes may also be used. A carrier structure 120 comprising, e.g., polyurethane foam as the carrier material, forms a solid gel grid with the solid gel electrodes. The carrier structure comprises, e.g., a stretchable carrier material, such as the polyurethane foam, for example. The electrodes of the plurality of electrodes are connected, e.g., to cables, and the cable routing runs on the upper side of the plaster surface. In other words, the cable are guided, with a clearance, along a surface of the carrier structure 120 facing away from the electrode arrangement 110. The clearance 140 in the cable routing limits a force transmission of a stretching of the carrier structure 120 to the cables. The clearance corresponds to, e.g., a length allowance in the cable length, e.g., in the form of a slung course of the cables. The carrier structure is configured, e.g., to extend a distance between two electrodes of the electrode arrangement by a maximum of 20%. The embodiment shown in FIG. 7 comprises a carrier structure 120 and an electrode arrangement 110 as it is also shown in FIG. 4b. The lugs at certain electrode positions serve for better adhesion, although they could also be removed. Alternatively, however, the embodiment shown in FIG. 4a may also be formed as a solid gel grid (carrier structure with commercially available solid gel electrodes, connected to cables). Optionally, the device 100 comprises a signal output 160 that is configured to provide an output signal based on the electric signals measured by the plurality of electrodes 110₁ to 110₉. The signal output 160 is connected to the electrodes of the electrode arrangement via cables. The cables go beyond, e.g., the carrier structure 120 and end in a plug acting as the signal output 160.

A second variant of the device 110 comprises a trace grid. In this embodiment, e.g., traces comprising a metal material (e.g. silver and/or Ag/AgCl) or a graphene material are applied to a carrier material of the carrier structure 120, e.g., by means of an etching method, a laser method, or also a printing method. It is also possible to use a metal combination, to first produce the traces in a copper etching method and subsequently silver-plate the electrode surfaces by means of chemical bonding. Printed (or etched) electrode systems on film carriers are advantageous as they can be stuck to the skin. Additionally, adhesive solutions, i.e. printed traces on a carrier material that can be stuck to the skin, are more robust against artefacts due to movement of the electrodes on the skin. The carrier material is, e.g., a semi-flexible, i.e. stretchable or elastic, thin film-like material (e.g., a polymer material, such as a polyester material or polyamide material, or a polyurethane material, or a silicone material). Polyurethane material is of advantage as it allows, in addition to skin-friendliness as a biocompatible material, for a stretchability and elasticity and a flexibility in more than one dimension. Thus, the grid, i.e. the carrier structure 120, fits snug on any unevenness on the face and head. For insulation, the traces are layered, e.g., as a sandwich construction with a second layer of the carrier material of the carrier structure 120, wherein the electrode surfaces (shown as circles in FIGS. 4a and 4b) remain exposed. The traces comprise, e.g., a slung course on or in the carrier structure 120 (e.g., wavelike, meandering or jagged) in order to extend correspondingly upon stretching of the carrier structure 120. The carrier structure is thus configured to extend a distance between two adjacent electrodes of the electrode arrangement by a maximum of 10%.

A principle sketch of the structure is shown in FIG. 8, wherein A is polyurethane, C is copper, and B is the silver-plating. The carrier structure 120 of the device 100 therefore comprises, e.g., a first layer of carrier material, a second layer of carrier material, and an interposed layer comprising a conductive material, such as a copper material, for example. At the positions on the carrier structure 120 at which the electrodes of the electrode arrangement are arranged, the second layer of carrier material comprises a gap. At these locations, the conductive material of the interposed layer is, e.g., silver-plated or covered by the graphene material-Subsequently, e.g., a gel, such as an adhesive electrolyte gel or a solid gel, is applied and/or pre-applied to the silver-plated location on the carrier structure 120. FIG. 8b shows the structure after pre-application of the gel (D), however, without a coverage by an optional protective film. It is important that the gel does not come into contact with the copper trace as, otherwise, chemical reactions will occur. The gel material arranged on the silver-plated electrode surface may be referred to, e.g., as a gel lens. The free electrode surfaces (shown as circles in FIGS. 4a and 4b) have a gel lens placed thereon. This is exposed by the person themselves, e.g., by peeling off a protective film directly before applying the grid. The protective film portions are subdivided, e.g., such that it is possible to stick on the arms of the grids one after the other. Optionally, an application of the gel to the electrode location may be omitted) the plurality of electrodes therefore comprises, e.g., dry electrodes) if a skin-friendly material such as graphene is used for covering the conductive material that is arranged between the two layers of carrier material.

If necessary, a stiffening element 122, e.g., comprising polyurethane foam may be attached as an additional stabilizer in this second variant and optionally also in the first variant. Alternatively, a sub-millimeter thick adhesive surface may also be sufficient, however. Also for stabilizing the application of the grid, a paper or film material may be attached on the upper (not skin) side, i.e. on the side of the carrier structure 120 facing away from the electrode arrangement 110, to increase the stiffness. This material is removed after placing the grid, similar to a shower bandage, so that overall, a wear comfort is higher.

According to an embodiment, the carrier structure 120 comprises transmissive carrier materials, i.e. transparent and/or colorless carrier materials, or differently colored carrier material in order to achieve a low (or, if necessary, particularly high) visibility of the carrier structure 120 for different types of skin. The narrow grid design is suitable to be worn in day-to-day life, if necessary with an optically pleasing print, or to remain unassuming due to the transparent design. The device may therefore be discrete and offer a pleasant wear comfort so that even an application in day-to-day life is made possible. Thereby, long-term recordings are made possible as an increased acceptance for a use of the device in day-to-day life is achieved. Transmissive carrier materials are particularly advantageous as these allow for fast detection of skin irritation, e.g., reactions to an adhesive for affixing the carrier structure on the skin. Additionally, transmissive carrier materials facilitate the autonomous application of the carrier structure as distinctive face points are not covered and a user can thus efficiently position the electrodes of the electrode arrangement correctly on themselves. Additionally, after application of the electrode arrangement, it is easy to determine whether the electrodes were positioned at the correct positions of the user.

According to an embodiment, the electrode arrangements 110 of the device 100 described herein are conceivable with passive or active electrodes. Passive electrodes derive the electric activity via a metal surface, in the example of the “variant solid gel grid”, e.g., via a metal platelet made of silver with a silver chloride coating (Ag/AgCl electrodes), and in the example of the “variant trace grid”, it is a silver surface that was applied to a copper trace in a chemical process. As a different type of electrodes, active electrodes exist that achieve an improvement of signal quality even before input into the difference amplifier using specific circuits. This additional circuit may serve different purposes. One possibility is the impedance conversion. Similar to a microphone cable, this allows, e.g., making the signal less sensitive to disturbances by movements of the cables. With the aid of this additional circuit, it is also possible to add small amplifiers already directly in the electrode. Thereby, the signal-noise ratio can be improved directly at the electrode, leading to better signal quality. Moreover, a signal amplifier, which the device optionally comprises, may be located further away on the body as movements of the cables are less critical to signal quality due to the better signal-noise ratio.

With very large or very small head shapes (elementary school children, teenagers, adolescents), the configuration is scalable, for example to 70% of the normal size for elementary school children. Oriented at international hat sizes regarding the head circumference, sizes S, M, L may be covered by the proposed carrier structure, i.e. head circumferences of 55-56 cm in S, 57-58 cm in M, and 59-60 cm in L. Due to the elastic carrier material, the carrier structure is adapted for different head circumferences, wherein the different head circumferences may have a maximum difference of 5 cm. Moreover, the configuration should be increased in size with a factor of 1.1 or 1.2 so that head sizes CL of 61-62 may also be covered. A further corresponding increase in size beyond this is conceivable. Likewise, the configuration may be adapted by a scaling by the factor 0.9 or 0.8 for sizes XS of 53-54 cm. Due to the softness of the carrier material of the carrier structure 120, it is unproblematic if the carrier structure 120 makes a small wave between the electrode positions if the head is slightly too small. Similarly, intermediate passages can also be stretched slightly to reach the positions on the forehead and behind the ear. An adaptation to different head shapes is possible, e.g., in a manner that the arm behind the ear is slightly twisted/tilted. The flexible material makes this possible. Advantageously, the traces are printed in a slung shape, e.g. in a meandering shape, in areas that are twisted or stretched, so that the traces can absorb the stretching and/or tension. The carrier structure is adapted, by means of the elastic carrier material, to positioning the at least one part of the electrode arrangement on the face surface of the head of the user with a head circumference in a range of 50 cm to 55 cm, or 55 cm to 60 cm, or 60 cm to 65 cm.

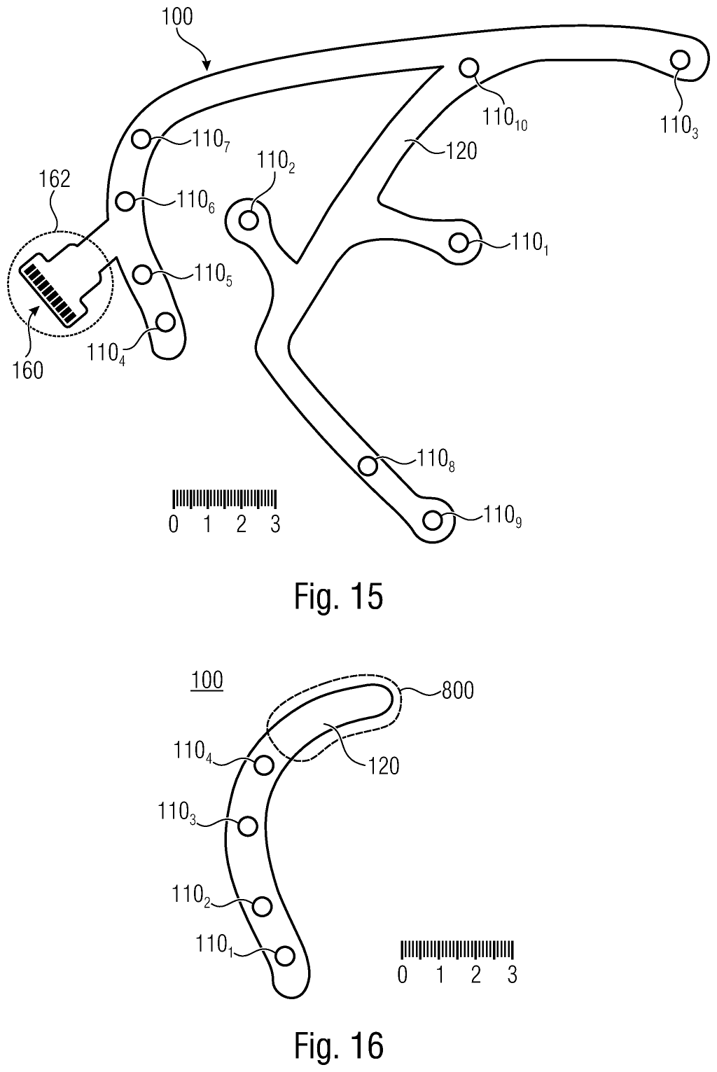

According to an embodiment, the device 100 comprises a signal amplifier 150 that is configured to receive the measured electric signals from a signal output (e.g. 160 in FIG. 7 or FIG. 15) of the device 100. The signal output 160 may optionally be arranged on the carrier structure 120, cf., e.g., FIG. 15. FIG. 15 shows, e.g., a trace grid in which the electrodes 110₁ to 110₁₀ are connected to the signal output 160 via traces. The traces are, e.g., integrated into the carrier structure 120. The device 100 further comprises, e.g., a fastening means defining a position of the signal amplifier on a back of the head, an arm, a shoulder, a chest, or a neck of the user. The device 100 comprises, e.g., an EEG difference amplifier for signal detection. To avoid cable artefacts, the amplifier should be placed near the electrodes. It is also possible to first lead to an adapter to then have a shielded cable lead to the amplifier. Then, the amplifier may also be worn using a chest belt or an armband. In mobile EEG applications in the field of research, it is often worn at the back of the head by means of a headband, which solution is also generally conceivable here. The EEG amplifier records the signals either locally or transmits them in a wired or wireless manner, e.g., via a Bluetooth connection, to a receiving station.

For connecting the device 100 of FIG. 15 to an adapter or directly to the signal amplifier 150, usually, a shape-stable/rigid reinforcement is needed as, otherwise, the connection cannot be established by insertion. For this purpose, a rigid material may be stuck on the plug-in piece 162 of the carrier structure 120 as a support. The carrier structure 120 comprises, e.g., a support structure, which is arranged, e.g., at a position 162 of the signal output 160 of the carrier structure 120. The carrier structure comprises, e.g., polyimide material, polyester material, polyethylene material and/or FR4 material.

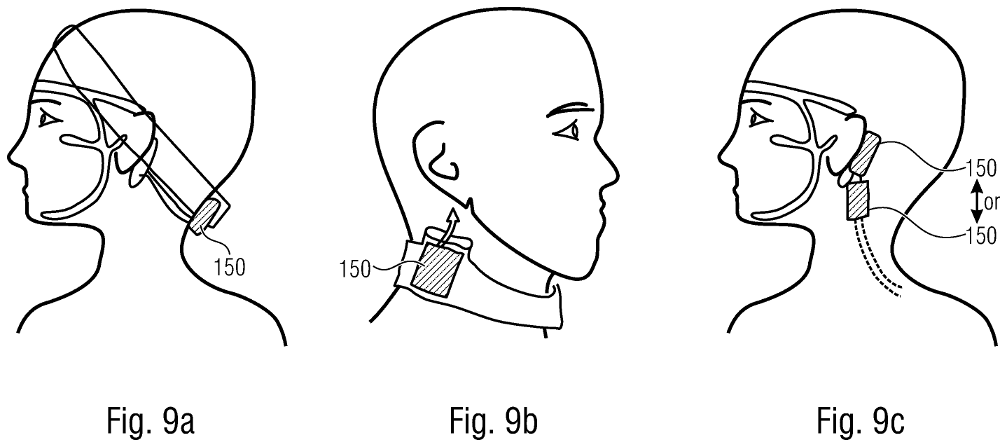

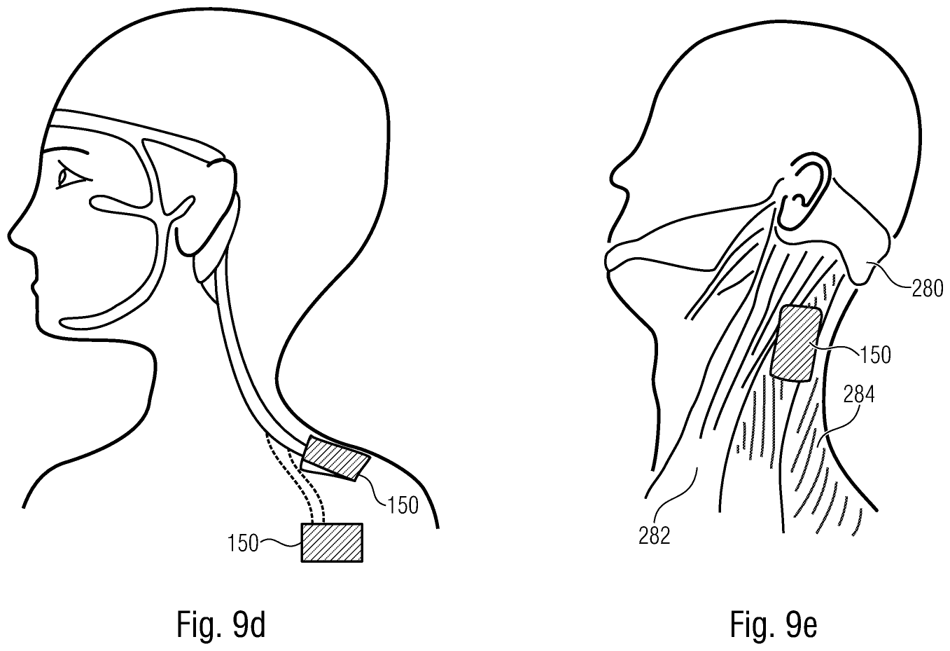

The signal amplifier 150 could be worn on the shoulder or at chest height by fastening it on clothing or a retaining strap. FIGS. 9a to 9e show different options of how the amplifier may be advantageously worn on a body of the user. FIG. 9a shows a possible positioning of the connection to the amplifier or adapter on the back of the head of the user using a headband. Another possibility is the placement on the neck of the user. The adapter or directly the amplifier could, e.g., be stuck to the skin by means of a skin-friendly adhesive patch. Another possibility is using a scarf or loop worn around the neck, as it is shown in FIG. 9b. Here, the adapter or amplifier may be accommodated in a bag on said scarf or loop. The scarf or loop may additionally serve for cushioning. As an alternative to the scarf, a retaining element around the base of the neck may also come to lie on the torso. In FIG. 9c, two alternatives are represented; the signal amplifier 150 may be arranged or fastened either directly behind the ear or, alternatively, below the ear. According to FIG. 9d, the signal amplifier 150 may be placed on the shoulder of the user are fastened to clothing or a retaining strap or, e.g., fasted to the chest of the user or their clothing or a retaining strap. Particularly for the application during sleeping, however, the position for the amplifier or adapter shown in FIG. 9e is of advantage. The fastening means is, e.g., a collar or scarf configured to affix the signal amplifier 150 on the neck of the user below the bony projection, the mastoid process 280, and between the two muscles sternocleidomastoid 292 and trapezius 284.

In the following, the use of the device 100 according to the invention is elaborated further, while it is particularly elaborated on how the user may apply the device 100 themselves. In this, the device 100 corresponds to, e.g., one of the previously described embodiments.

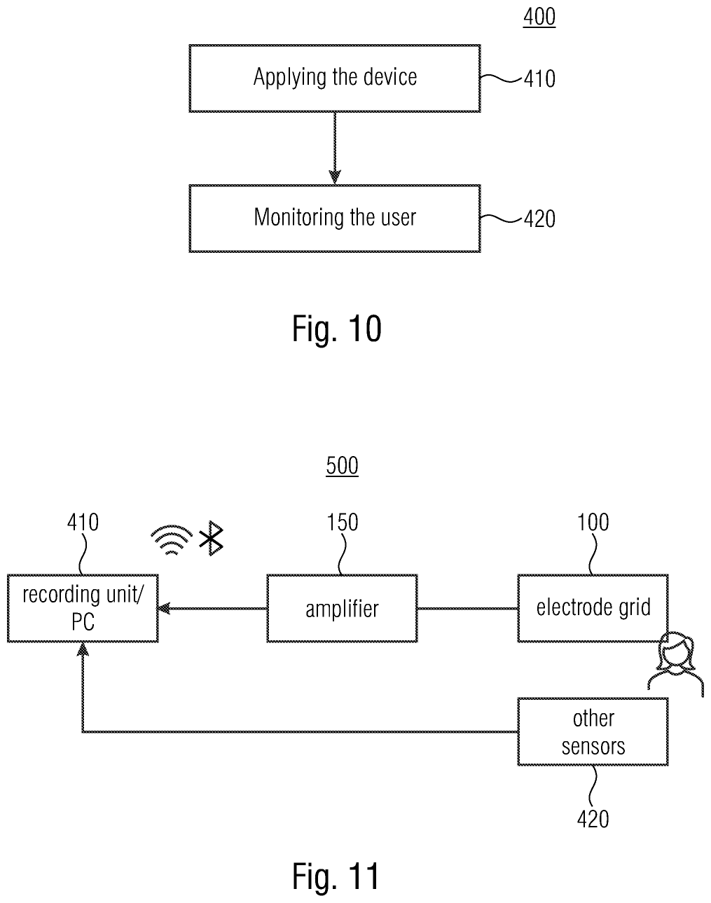

As shown in FIG. 10, the method 400 for using the device according to the invention comprises at least the steps of applying 410 the device 100 on a face surface 220 of a head 200 of a user 210 and/or behind an ear 230 of the user by the user 210 themselves or on a different person, and monitoring 420 the user 210 by means of the signals measured by the plurality of electrodes. For the application 410, an autonomous application 410 by the user 210 is of advantage.

Optionally, the method 400 may comprise further ones of the steps described below:

The method may comprise cleaning the head surface, i.e. the skin, wherein cleaning is optionally limited to the areas on which the device is applied 410. Cleaning the skin using an alcoholic solution for removing dirt, grease, and sebum for better signal quality is advantageous. Alternatively or additionally, cleaning, e.g., the pretreatment of the head surface, may be carried out using an abrasive gel, as it is often common with ring electrodes. A technical problem is the low signal strength of the potential difference in the range of 0.1 to 100 microvolt. For this reason, the contact between the electrode and the skin must be established with the best possible conductivity. The impedance value of the skin falls in the range of a few kOhm to MOhm. In order to keep this value low, the locations at which the electrodes are positioned are, e.g., degreased and, if necessary, treated with an exfoliating gel before the measuring to remove grease, sebum, or dead skin cells.

In one embodiment of the device 100, the carrier structure 120 comprises adhesive material that is covered, e.g., by a protective film. The bottom side of the carrier structure, i.e. the side of the carrier structure on which the electrode arrangement is arranged is, e.g., covered by the protective film under which an adhesive surface of the adhesive material is located on the grid arms, i.e. on the connecting ribs of the carrier structure. At the electrode positions, e.g. a gel lens for the advantageous signal connection between the skin and the electrode is arranged. Before the protective film is peeled off for fastening the device on the skin, a preplacement takes place, for example. Applying 410 the device may therefore comprise, e.g., the steps of
preplacing the device on the head surface, i.e. on the face surface 220 and behind the ear 230,
peeling the protective film off the device, and
sticking the device onto the head surface.

Peeling off and sticking on may be repeated, e.g., multiple times if the device comprises a plurality of protective film portions. The order of when which protective film portion is peeled off may take place, e.g., as already described above in the context of the device.

Via the two electrodes A and B (references in FIG. 5a and FIG. 5b), e.g., the preplacement takes place by feeling the upper cheek bone, preplacing the electrode B thereon, and preplacing the electrode A in an approximately horizontal line. Then, it may be checked whether the upper grid arm can be placed above the eye, at the approximate center of the forehead (C). Likewise, it can be checked whether the left arm of the grid (D) fits well behind the ear. For an easy handling, the design therefore selected the course of the arm above the ear as this offers a natural support on the upper base of the ear on the head during the placement. By means of a slight rotation about point F, the positions of the left and upper arm (i.e. the positions of the two parts 124₁ and 124₂ in which the second connecting rib 124 branches off) may be optimized. With one free hand and a skin friendly pen, the positions of (A) and (B) may additionally be visibly marked on the face so that a correction is easily possible even if the hand slips.

After the preplacement, the protective films can be peeled off starting at the arm with the electrodes A and B in order to then stick on the arms, i.e. the connecting ribs of the carrier structure, of the grids in the order (C, D) named during preplacement. The protective film is subdivided into corresponding portions so that the arms of the grid can be attached individually one after the other.

Applying 410 the device 100 on the face surface 220 of the head 200 of the user 210 can thus comprise, e.g., the steps of feeling an upper cheek bone and affixing a first electrode on the upper cheek bone; affixing a second electrode in front of the tragus of an ear of the user; affixing a third electrode on a forehead, vertically above a nasion in an area of 1-3 cm above an eyebrow line; and affixing a fourth electrode behind the ear.

For describing the placement:
1. Feeling the cheekbone and a hair free area (important for use on people with a beard) in front of the ear (about 1 cm in front of the tragus “cartilage bump”). Placing the two short branches of the grid on these points, in an approximately horizontal orientation.
2. Orienting the grid roughly on the forehead and behind the ear, if necessary, rotating about point F
3. Sticking the grid on piece-by-piece, beginning at the cheek bone and in front of the tragus.
4. On the forehead, the point is hit vertically above the nasion towards the forehead, at about 1-2 cm above the eyebrow line. A horizontal deviation in a range of 1-2 cm is acceptable.
5. Behind the ear, the last electrode usually reaches the mastoid as a palpable bony projection. This electrode is typically selected as a reference for the measurement of potential differences.

In an optional embodiment of the grid, a third arm (E) exists, which is stuck on along the chin in order to record via the electrodes for an EMG in the chin area. Such a recording is advantageous in detecting sleep phases for distinguishing the rapid eye movement (REM) phase from the other phases, particularly the waking phase. The complete grid design with the electrodes on the chin for detecting an EMG is targeted at the application for sleep monitoring in a domestic environment.

The good handling of the grid and the described possibility of application ensures a secure placement on the first try. In the case of re-placing, adhesive strength would get lost, increasing the risk of the signal stability not being maintained over longer periods.

The hook region above the ear is, e.g., spared as an adhesive surface so that the grid does not stick to hair. In addition to helping with placing the grid, this hook additionally serves the function as a connection between the face part and the arm behind the ear. A different embodiment variant below the ear is possible, which would run along the jawbone, which compromises the stability of the electrode placement in the case of increased jaw activity and furthermore offers less wear comfort.

For gathering electrophysiological signals, particularly electroencephalograms (EEG), electrooculograms (EOG), electrocardiograms (ECG or EKG), and electromyograms (EMG), the electrodes are connected to the skin of the human via a gel (wet electrodes) or also without gel (dry electrodes) to measure electric activity generated by the brain and muscle cells. Potential fluctuations of an electrode are measured in difference from a reference electrode, the amplification of the signal takes place, e.g., by means of a difference amplifier. When using wet electrodes, an electrolyte gel is used for reducing the transition impedance between the skin and the electrode.

By means of the step of monitoring 420 the user, a sleeping behavior of the user, or an epileptic behavior of the user, or a neurological disorder of the user, or a cognitive psychological behavior of the user can be monitored, e.g., based on the measured electric signals.

Technical fields of application as they will be described below are conceivable:
domestic or outpatient sleep monitoring
domestic or outpatient monitoring in patients with epilepsy or suspected epilepsy
domestic or outpatient monitoring in patients with other neurological disorders or suspected disorders (e.g. depression, Alzheimer's), inter alia, for deriving early indicators.
the use in cognitive psychological test series, for example, regarding attention, vigilance, hearing effort, cognitive stress, determining sensory functionality and cognitive processing ability of the patient in a domestic environment, in care facilities, but also at doctor's offices or in hospitals and clinics since there, as well, the simple installation of the electrodes saves time
use for detecting the aforementioned cognitive conditions in work situations, in particular for safety-critical applications (e.g. train conductors, pilots, air traffic controllers, truck drivers, mining vehicles)
in neuropsychological research and in adjacent research fields regarding cognitive conditions, but also regarding neurological disorder symptoms in a waking but also in a sleeping state
detecting the acoustic attention for subsequent analyses or for controlling a hearing aid, for example for improving the speech signal of an attending speaker
detecting the hearing effort for subsequent analysis or also for controlling the settings of a hearing aid
monitoring the effectiveness of positive air pressure systems for treating apnea. Positive air pressure (PAP) systems are one of the therapies for apnea. Usually, first, an apnea disorder is diagnosed in a sleep laboratory, then the PAP device is set up, also in a sleep laboratory. In practice, however, this set up supposedly does not work sufficiently well in many cases, in which the sleep is usually merely tested at intervals using a polygraph (without EEG). The solution proposed herein could bring about an improvement as the detection continues to be possible in the domestic environment while allowing for higher quality information on the sleep architecture by means of an EEG.

FIG. 11 shows a block diagram for the structure of data acquisition using the device 100. People may take a system 500 for data acquisition home and apply it there themselves. The system comprises, e.g., an EEG amplifier 150, a recording unit 410, and, if necessary, further sensors 420 and the device 100 according to the invention as described herein.

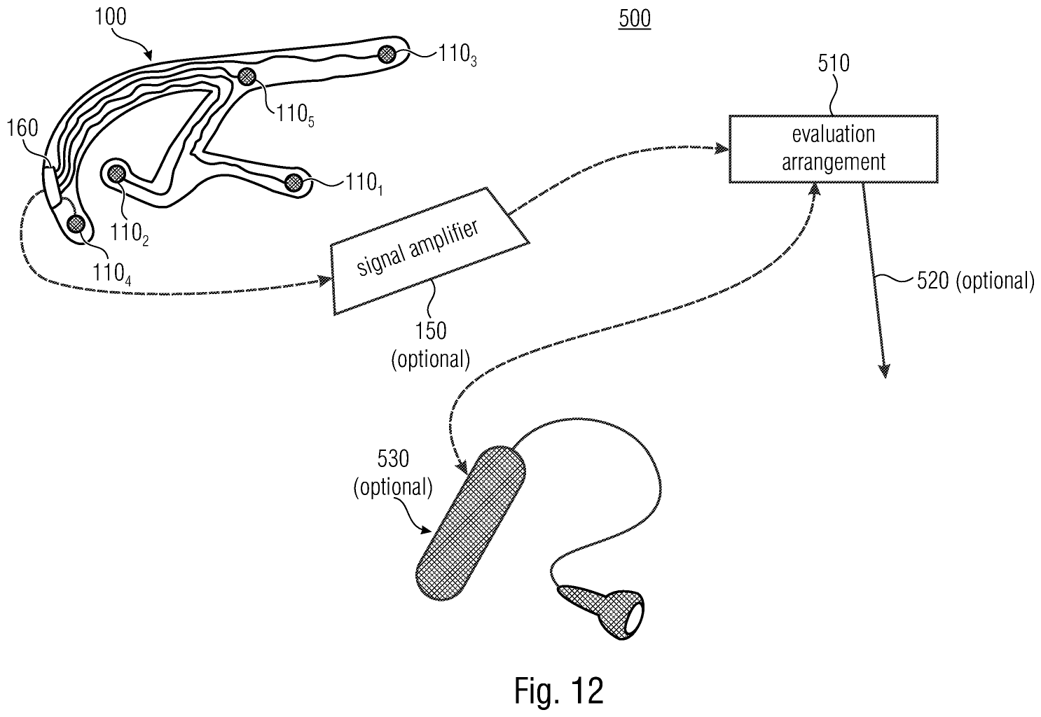

FIG. 12 also relates to an embodiment of a system 500 comprising the device 100 according to the invention. Additionally, the system 500 comprises an evaluation arrangement 510 that is configured to evaluate the electric signals measured by the plurality of electrodes. Optionally, the system 500 may comprise features and functionalities as they have been described in the context of FIG. 11.

The evaluation arrangement 510 is configured, e.g., to receive the electric signals measured by the plurality of electrodes from a signal output 160, e.g., via a signal amplifier 150. This may take place, e.g., wirelessly via WLAN or Bluetooth, or also in a wired manner.

The evaluation arrangement 510 is configured, e.g., to provide an information 520 on a sleep architecture of the user based on the electric signals; or to provide an information 520 on an epileptic behavior of the user; or to indicate a neurological disorder of the user 520; or to provide a cognitive psychological information 520.

According to an alternative embodiment, the system 500 further comprises a hearing aid 530. In this case, the evaluation arrangement is configured, e.g., to control the hearing aid 530 based on the electric signals.

The evaluation arrangement 510 may be configured, e.g., to:
detect an acoustic attention for subsequent analyses and/or for controlling the hearing aid 520, for example for improving the speech signal of an attending speaker; and/or
detect the hearing effort for subsequent analysis and/or also for controlling the settings of the hearing aid 530.

The added value of the solution proposed herein is that, based on the self-applicable device 100 in the prescribed configuration of the electrodes 110₁ to 110₅ (the configuration of the electrodes may be present according to an embodiment described herein, cf., e.g., FIGS. 1a, 4a, 4b, and 6a/b), in addition to EKG, EMG, advantageously for the proposed application EEG and EOG information may be gathered in day-to-day life and may thus be connected to the hearing aid 530 and optionally to an EEG/EOG signal amplifier. The device 100 in the system 500 is particularly advantageous if the electrode arrangement comprises wet electrodes. Solutions with dry electrodes in the ear canal or in the outer ear exist, although these may cause unpleasant pressure or only offer worse signal quality. The solution proposed herein combines wear comfort and good signal quality. If an EEG can be gathered in day-to-day life in this form, the mentioned applications in connection with a hearing aid are conceivable, these are:
detecting the acoustic attention to a particular speaker in a situation with multiple speakers (or to a noise in a loud environment) based on the EEG via a correlation of EEG and the envelope of the target audio signal (scientific works regarding this have been published for a few years). If the target signal is detected this way, the beam former of the hearing aid may subsequently be set such that this target signal is particularly highlighted and is therefore more audible for the hearing person
in the previously designated application case, it is a particular challenge to allow the hearing person to also “get out of” the supported situation, meaning to enable the hearing person to also focus on other speaker sources again, although these are now further in the background. A solution based on the proposed electrode grid is to receive information, via the EOG, on eye movement (direction), blinking, and closing eyes and to use these in a way as a remote control. This way, the hearing person may control in a much more robust and simultaneously a simple and intuitive manner, which signal is desired to be particularly highlighted (look in the direction, blink twice for confirmation, for example). The evaluation arrangement 510 may thus be configured to evaluate the signals measured by the device 100 for predetermined signal properties to control the hearing aid 530 in response to signals of the user.
detecting the hearing effort based on EEG data—if the hearing effort increases, the hearing aid algorithms may be adapted automatically, for example to another preprogrammed profile of the hearing support algorithms. However, here, the analysis of the audio context may also offer an additional information source for selecting the hearing support algorithm.

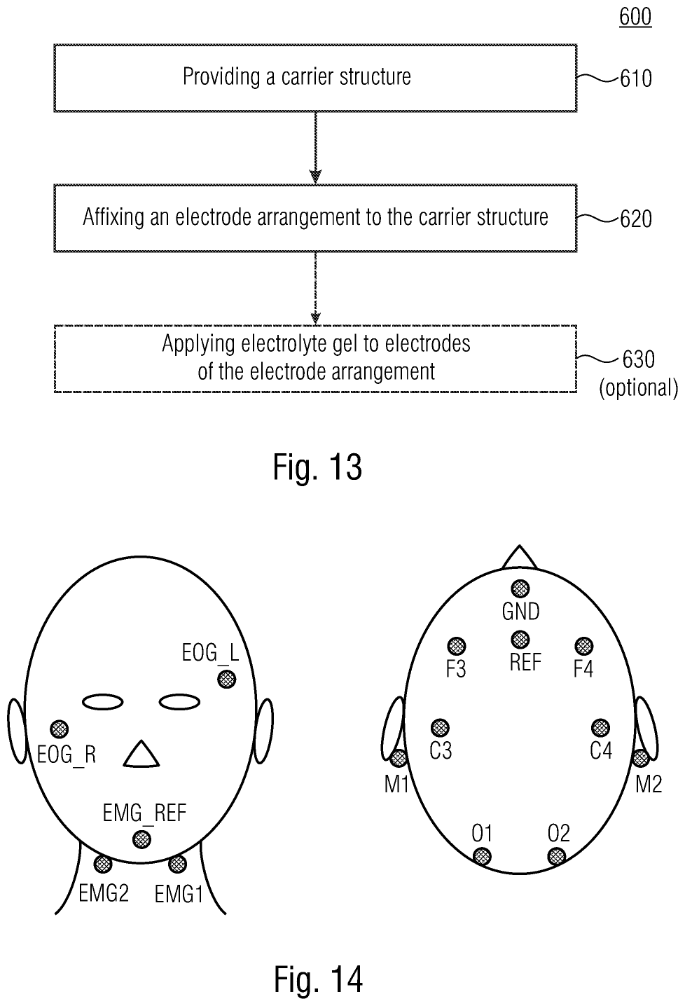

A further embodiment relates to a method 600 for producing the device 100 according to the invention, see FIG. 13. The method 600 comprises, inter alia, the steps of providing 610 a carrier structure 120 and affixing 620 an electrode arrangement 110 on the carrier structure 120, and optionally applying 630 electrolyte gel or gel lenses to electrodes of the electrode arrangement. The electrode arrangement comprises, e.g., a plurality of electrodes. Upon application 630, one gel lens and/or electrolyte gel each may be applied 630, e.g., only on a part of the plurality of electrodes, or one gel lens and/or electrolyte gel may be applied 630 to each electrode of the plurality of electrodes.

The electrode arrangement comprises a plurality of electrodes for measuring electric signals on a face surface of a head and/or behind an ear of a user of the device.

The carrier structure is adapted to a face surface of the head of the user of the device. The carrier structure is shape-stable within a tolerance range without external force exerted by the user and deformable under external force exerted by the user in order to change a distance between to adjacently arranged electrodes, and/or to area-comprehensively adapt to face and/or head structures. Furthermore, the carrier structure defines a relative position of the electrodes of the plurality of electrodes to one another. The carrier structure is configured to position at least a part of the electrode arrangement on the face surface and/or behind the ear. The plurality of electrodes of the electrode arrangement may be subdivided, e.g., in a first subset of electrodes and a disjoint second subset of electrodes, wherein the carrier structure is configured, e.g., to position the first subset of electrodes on the face surface, and to position the second subset of electrodes behind the ear.

Affixing 620 the electrode arrangement may take place, e.g., as already described in connection with the device 100. Particular reference is made to the embodiments in FIGS. 7, 8a, and 8b. Thus, fully manufactured wet or dry electrodes may be stuck to the carrier structure as shown, e.g., in FIG. 7, or the electrode arrangement may be integrated into the carrier structure as shown in FIGS. 8a and 8b. An integration of the electrodes into the carrier structure is particularly advantageous if they are connected to traces. A sandwich structure as shown in FIGS. 8a and 8b, protects the user of the device, as the traces are not skin-friendly and is used for electric insulation of the traces.

As an alternative to the previously described embodiments of the device 100 according to the invention, it may comprise a carrier structure 120 as shown in FIG. 16 that is configured to position the plurality of electrodes 110₁ to 110₄ of the electrode arrangement 110 only behind the ear 230. For this purpose, the carrier structure 120 comprises, e.g., a part 800, such as a hook, that is configured to be supported on the ear of the user while positioning and/or affixing the device 100 behind the ear. This allows for autonomously affixing the device 100 behind the ear of the user.

The electrodes 110₁ to 110₄ may comprise, e.g., the same features, positions, and/or functionalities as the electrodes 110₄ to 110₇ in FIG. 4a. Optionally, an extension 130 as it is shown, e.g., in connection with the electrodes 110₄ and/or 110₅ in FIG. 4b, may be arranged on the electrode 110₁ and/or on the electrode 110₂. The electrodes 110₁ to 110₄ may be connected, e.g., to traces or to cables.

The plurality of electrodes 110₁ to 110₄ of the electrode arrangement 110 are configured for measuring electric signals behind the ear of the user. Advantageously, the electrode arrangement comprises a plurality of wet electrodes. The electrode arrangement 110 is arranged on or integrated in the carrier structure 120, for example.

The shape of the carrier structure 120 follows, e.g., the shape of the ear of the user. The carrier structure 120 at least partially defines a relative position of the electrodes of the plurality of electrodes to one another. The shape and length of the carrier structure is configured, e.g., to lead from an upper base of the ear, along the ear, to a mastoid. This is particularly advantageous for a self-application of the device 100 as the user may very exactly feel either the upper base of the ear or the mastoid and may thus position and affix the device and/or the carrier structure 120 behind the ear.

The carrier structure 120 is shape-stable within a tolerance range without external force exerted by the user and deformable under external force exerted by the user in order to change a distance between to adjacently arranged electrodes, and/or to area-comprehensively adapt to face and/or head structures.

Furthermore, it is to be noted that the device 100 may comprises features and functionalities as they have been described in the context of other embodiments of the device 100 according to the invention, cf., e.g., FIG. 1a to FIG. 9e. In particular, the carrier structure 120 may comprise, e.g., extensions 130 on electrodes of the plurality of electrodes. Furthermore, the carrier structure 120 may comprise, inter alia, features such as adhesive material, protective films, and stiffening elements.

Even though some aspects have been described within the context of a device, it is understood that said aspects also represent a description of the corresponding method, so that a block or a structural component of a device is also to be understood as a corresponding method step or as a feature of a method step. By analogy therewith, aspects that have been described within the context of or as a method step also represent a description of a corresponding block or detail or feature of a corresponding device. Some or all of the method steps may be performed by a hardware device (or using a hardware device), such as a microprocessor, a programmable computer, or an electronic circuit, for example. In some embodiments, some or several of the most important method steps may be performed by such a device.

Depending on specific implementation requirements, embodiments of the invention may be implemented in hardware or in software. Implementation may be effected while using a digital storage medium, for example a floppy disc, a DVD, a Blu-ray disc, a CD, a ROM, a PROM, an EPROM, an EEPROM or a FLASH memory, a hard disc or any other magnetic or optical memory which has electronically readable control signals stored thereon which may cooperate, or cooperate, with a programmable computer system such that the respective method is performed. This is why the digital storage medium may be computer-readable.

Some embodiments in accordance with the invention thus comprise a data carrier, which comprises electronically readable control signals that are capable of cooperating with a programmable computer system such that any of the methods described herein is performed.

Generally, embodiments of the present invention may be implemented as a computer program product having a program code, the program code being effective to perform any of the methods when the computer program product runs on a computer.

The program code may also be stored on a machine-readable carrier, for example.

Other embodiments include the computer program for performing any of the methods described herein, said computer program being stored on a machine-readable carrier.

In other words, an embodiment of the inventive method thus is a computer program which has a program code for performing any of the methods described herein, when the computer program runs on a computer.

A further embodiment of the inventive methods thus is a data carrier (or a digital storage medium or a computer-readable medium) on which the computer program for performing any of the methods described herein is recorded. The data carrier, the digital storage medium or the computer-readable medium are typically tangible or non-volatile.

A further embodiment of the inventive method thus is a data stream or a sequence of signals representing the computer program for performing any of the methods described herein. The data stream or the sequence of signals may be configured, for example, to be transmitted via a data communication link, for example via the internet.

A further embodiment includes a processing unit, for example a computer or a programmable logic device, configured or adapted to perform any of the methods described herein.

A further embodiment includes a computer on which the computer program for performing any of the methods described herein is installed.

A further embodiment in accordance with the invention includes a device or a system configured to transmit a computer program for performing at least one of the methods described herein to a receiver. The transmission may be electronic or optical, for example. The receiver may be a computer, a mobile device, a memory device or a similar device, for example. The device or the system may include a file server for transmitting the computer program to the receiver, for example.

In some embodiments, a programmable logic device (for example a field-programmable gate array, an FPGA) may be used for performing some or all of the functionalities of the methods described herein. In some embodiments, a field-programmable gate array may cooperate with a microprocessor to perform any of the methods described herein. Generally, the methods are performed, in some embodiments, by any hardware device. Said hardware device may be any universally applicable hardware such as a computer processor (CPU), or may be a hardware specific to the method, such as an ASIC.

For example, the apparatuses described herein may be implemented using a hardware device, or using a computer, or using a combination of a hardware device and a computer.

The apparatuses described herein, or any components of the apparatuses described herein, may at least be partially implement in hardware and/or software (computer program).

For example, the methods described herein may be implemented using a hardware device, or using a computer, or using a combination of a hardware device and a computer.

The methods described herein, or any components of the methods described herein, may at least be partially implement by performed and/or software.

While this invention has been described in terms of several embodiments, there are alterations, permutations, and equivalents which fall within the scope of this invention. It should also be noted that there are many alternative ways of implementing the methods and compositions of the present invention. It is therefore intended that the following appended claims be interpreted as including all such alterations, permutations and equivalents as fall within the true spirit and scope of the present invention.

1. A device comprising:
an electrode arrangement with a plurality of electrodes for measuring electric signals on a face surface of a head of a user and behind an ear of the user, wherein the electrodes of the plurality of electrodes are wet electrodes;
a carrier structure;
wherein the carrier structure at least partially defines a relative position of the electrodes to one another; and
wherein the carrier structure is shape-stable within a tolerance range without external force exerted by a user and deformable under external force exerted by the user, in order to change a distance between two adjacently arranged electrodes and to area-comprehensively adapt to face and head structures,
wherein the carrier structure is configured to position at least a part of the electrode arrangement on the face surface and behind the ear;
wherein the carrier structure is configured in the shape of a net structure with branching connecting ribs that follow a course of the face surface; and
wherein the branched connecting ribs comprise a rib width corresponding to, at most, three times the electrode diameter of an electrode of the plurality of the electrodes.

2. The device according to claim 1, wherein the carrier structure follows the face surface as a human face shape, and the plurality of electrodes are arranged on the carrier structure;
wherein a first electrode position that is assigned to a first electrode of the plurality of electrodes corresponds to a position of a first distinctive point (B) of the face surface; and a second electrode position that is assigned to a second electrode of the plurality of electrodes corresponds to a position of a second distinctive point (A) of the face surface; and a third electrode position that is assigned to a third electrode of the plurality of electrodes results starting from the first electrode position and the second electrode position on the face surface within a tolerance range determined by the carrier structure.

3. The device according to claim 2, wherein the first distinctive point (B) of the face surface corresponds to an upper cheekbone, and the second distinctive point (A) corresponds to a position in front of the tragus of an ear of the user; and
wherein the carrier structure comprises a connecting rib that at least partially connects the first electrode position and/or the second electrode position, on one side, to the third electrode position, on the other side, and wherein the third electrode position corresponds, within a tolerance range of 1 to 2 cm determined by the carrier structure, to a position (C) on a forehead of the face surface in an area of 1 cm to 3 cm above the eyebrow line, vertically above a nasion of the user; and/or
wherein the carrier structure comprises a connecting rib that at least partially connects the first electrode position and/or the second electrode position, on one side, to a fourth electrode position that is assigned to a fourth electrode of the plurality of electrodes, on the other side, and wherein the fourth electrode position corresponds, within a tolerance range determined by the carrier structure, to a position (D) behind the ear of the user; and/or
wherein the carrier structure comprises a connecting rib that at least partially connects the first electrode position and/or the second electrode position, on one side, to a fifth electrode position that is assigned to a fifth electrode of the plurality of electrodes, on the other side, and wherein the fifth electrode position corresponds, within a tolerance range determined by the carrier structure, to a position (E) on the chin of the face surface of the user.

4. The device according to claim 1, wherein the carrier structure comprises an adhesive material for affixing the plurality of electrodes and the carrier structure on the face surface,
wherein the electrodes further comprise an adhesive electrolyte gel for lowering the impedance and for affixing the plurality of electrodes and the carrier structure on the face surface; wherein the adhesive electrolyte gel is covered, at least in some sections, by a removable protective film, and
wherein the adhesive material is covered by the removable protective film,
wherein the protective film comprises a plurality of protective film portions that are independently removable from the carrier structure and the plurality of electrodes.

5. The device according to claim 1, wherein a connecting rib of the branching connecting rib is curved at one end so that the carrier structure comprises a curved end piece that is configured to be affixed behind an ear of the user, and wherein the connecting rib is configured to run from the face of the user above the ear to the curved end piece, and wherein at least one electrode of the plurality of electrodes is arranged at the curved end piece.

6. The device according to claim 1, wherein a shape of the carrier structure is configured to position the plurality of electrodes at a corresponding plurality of positions on the face surface; wherein the plurality of positions is configured for a detection of the electric signals on the face surface for at least one linear combination of the electric signals for mapping signals from a predetermined head region of the user.

7. The device according to claim 1, wherein the carrier structure comprises polyurethane material, polymer material, and/or silicone material as a carrier material,
wherein the carrier material is elastic or stretchable;
wherein the electrodes are connected to conductive traces, and
wherein the conductive traces comprise a slung course on or in the carrier structure in order to correspondingly extend upon stretching of the carrier structure, and
wherein the carrier structure is configured to extend a distance between two electrodes of the electrode arrangement by a maximum of 10%.

8. The device according to claim 1, wherein on a side facing away from the electrode arrangement, the carrier structure comprises a removable stiffening element that is configured to at least locally stiffen the carrier structure.

9. The device according to claim 1, wherein the carrier structure comprises at least one extension comprising an adhesive material at a position at which an electrode of the electrode arrangement is arranged.

10. The device according to claim 1, wherein the plurality of electrodes comprises active electrodes comprising a circuit that is configured to
perform an impedance conversion of the measured electric signal, and/or
amplify the measured electric signal.

11. A system comprising:
a device according to claim 1; and
an evaluation arrangement that is configured to evaluate the electric signals measured by the plurality of electrodes.

12. The system according to claim 11, wherein the evaluation arrangement is configured to, based on the electric signals
provide an information on a sleep architecture of the user; or
provide an information on an epileptic behavior of the user; or
indicate a neurological disorder of the user; or
provide a cognitive psychological information.

13. The system according to claim 11, comprising
a hearing aid;
wherein the evaluation arrangement is configured to control the hearing aid based on the electric signals,
wherein the evaluation arrangement is configured to correlate the signals measured by the device with an envelope of an audio signal recorded by the hearing aid in order to control a beam former of the hearing aid.

14. A method for using the device according to claim 1, comprising:
applying the device on a face surface of a head of a user by the user themselves or by another person;
monitoring the user by means of the electric signals measured by the plurality of electrodes.

15. The method according to claim 14, wherein applying the device on the face surface of the head of the user comprises:
feeling an upper cheekbone and affixing a first electrode on the upper cheekbone;
affixing a second electrode in front of the tragus of an ear of the user;
affixing a third electrode on a forehead, vertically above a nasion, in an area of 1-3 cm above an eyebrow line;
affixing a fourth electrode behind the ear.
